(19) United States
(12) Patent Application Publication
(10) Pub. No.: US 2023/0043948 A1
(43) Pub. Date: Feb. 9, 2023

(54) METHOD AND KIT FOR CELL GROWTH

(71) Applicant: PRECISION CANCER TECHNOLOGIES INC ., Toronto (CA)

(72) Inventors: Simone RIZZI, Ecublens (CH); Jeremy TOUATI, Sottens (CH); Giulia FREGNI, Nyon (CH); Cara BUCHANAN PISANO, Préverenges (CH); Franck COUMAILLEAU, Lausanne (CH); Emanuele GAUDIELLO, Basel (CH); Sophie GRETTAZ, Diolly (Savièse) (CH); Nicolas CHARTIER, Eclagnens (CH); Mathieu HEULOT, Penthalaz (CH)

(73) Assignee: PRECISION CANCER TECHNOLOGIES INC ., Toronto, ON (CA)

(21) Appl. No.: 17/781,944

(22) PCT Filed: Dec. 1, 2020
(86) PCT No.: PCT/EP2020/084092

§ 371 Date: Jun. 2, 2022

(30) Foreign Application Priority Data
Dec. 4, 2019 (EP) 19213642.2
Jun. 4, 2020 (EP) 20178270.3

(51) Int. Cl. C12N 5/00; C12N 5/09; G01N 33/50

(52) U.S. Cl. CPC C12N 5/0075; C12N 5/0693; G01N 33/5008; C12N 2513/00; C12N 2533/52; C12N 2537/10; C12N 2539/00

(57) ABSTRACT

The present invention is related to a method to be performed with one tissue type, wherein a specific combination of hydrogel features has been pre-selected for the said one tissue type to be tested. The present invention is also related to a kit of parts to perform said method.

The present invention is related to a method and kit for cell growth that provides a significantly improved tool for drug discovery and development, but also for basic scientific research, precision medicine, regenerative medicine, and for delivery of cells for implantation into a mammal, preferably a human.

### BACKGROUND OF THE INVENTION

### Hydrogels for Cell Growth and Drug Screening

In the field of ex vivo assays, progress has been made in recent years. In particular the development of three-dimensional (3D) hydrogel matrices has provided a significant advantage over two-dimensional cell culture systems, which do not sufficiently resemble in vivo conditions.

First, naturally derived 3D cell culture systems such as Matrigel® were used. However, such systems have poorly defined compositions and show batch to batch variation, which makes it impossible to alter their properties in systematic ways and to independently control their key matrix parameters. Also, for screening purposes employing multi-well arrays, such naturally derived 3D cell culture systems are not well suitable, since due to their poor definition changes in the cellular behaviour between arrays cannot be precisely attributed to a specific modification of the extracellular matrix conditions provided in those arrays.

Also, the batch-to-batch variation and undefined composition of animal derived matrices such as Matrigel® prohibit regulatory approval for their use in humans. The development of a support matrix and of culture media are required that are defined and approved for human use, scalable, and preferably xeno-free (i.e. free from components of animal origin).

In recent years, however, fully defined semi-synthetic or fully synthetic hydrogel systems were developed that are much more suitable for the above purposes. For example, PEG-based hydrogels were described that are composed of PEG (polyethylene glycol) precursor molecules that are cross-linkable using either thrombin-activated Factor XIIIa under physiological conditions by a crosslinking mechanism that is detailed in Ehrbar et al. (Ehrbar, M., Rizzi, S. C., Schoenmakers, R. G., Miguel, B. S., Hubbell, J. A., Weber, F. E., and Lutolf, M. P., Biomolecular hydrogels formed and degraded via site-specific enzymatic reactions, Biomacromolecules 8 (2007), 3000-3007), or via mild chemical reactions by a crosslinking mechanism as detailed in Lutolf et al. (Lutolf, M. P., and Hubbell, J. A., Synthesis and physicochemical characterization of end-linked poly(ethylene glycol)-co-peptide hydrogels formed by Michael-type addition, Biomacromolecules 4, 713-722 (2003)). These PEG hydrogels are tuneable with respect to their properties and biocompatible.

Examples of applications of such fully defined semi-synthetic or fully synthetic hydrogel systems are in the field of transplantation into humans, basic research, precision medicine, drug discovery and development, for example in cancer research.

In cancer research and in the clinics, resistance to therapeutic treatments (e.g. against chemotherapy with cytotoxic substances and/or immunotherapy and/or targeted therapy) of the tumor cells is one major challenge.

Drug resistance of tumor cells to chemotherapy was usually attributed to genetic alterations and clonal genetic heterogeneity. However, mechanisms that leads to drug resistance in cancer cells are multiple (e.g. drug inactivation, cell death inhibition, DNA damage repair, drug target alteration, epithelial-mesenchymal transition, drug efflux, physical barriers, etc.) and can act independently or in combination and can be also dependent on epigenetic changes of cancer cells and on the influence of the tumor micro-environment (Holohan C, et al., 13, 714-726(2013)).

Indeed, tumors are generally composed of multiple phenotypic subpopulations that vary in their ability to initiate metastases and in their sensitivity to anticancer therapy (Flavahan et al., Epigenetic plasticity and the hallmarks of cancer, Science 357, 266 (2017); Baylin et al., Nat Rev Cancer, 11(10), 726-734 (2011)). In many cases, cells show transition between these subpopulations independently of genetic mutations, but instead through reversible changes in signal transduction and gene expression programs influenced by tumor-stroma cells, vasculature, immune system and the extracellular matrix (ECM) composition (Juntilla et al., Nature, 501(7467):346-54 (2013)).

Resistance to targeted therapy can be sub-classified as intrinsic resistance, adaptive resistance and acquired resistance. Intrinsic resistance might be due to driver mutations that are insensitive to therapy. Adaptive resistance occurs when, after a partial initial response to treatment, cancer cells undergo adaptive changes that allow their survival after the therapy. Acquired resistance can be the consequence of both selection for pre-existing mutations in a heterogeneous subpopulation (i.e. initially not all cancer cells in the tumor are dependent on the target) and the acquisition of new alterations (phenotypically or genetically) due to the selective pressure exerted by the therapy. Mechanisms of resistance can involve either the primary target of the drug or other signalling events that can bypass the target by inducing other survival and/or growth pathways (Rotow-Bivona et al., Nature Reviews Cancer, 17(11) 637-658(2017)).

Therefore, using a single condition for in vitro culture of a specific cancer might not be enough to maintain the heterogeneity necessary to represent ex vivo the different genetic and phenotypic tumor cell characteristics that may be responsible to different drug response and thus drug resistance.

Initially thought to be a passive support, the ECM, with its tethered bioactive domains and also as reservoir of soluble cytokines, is emerging as a key player in malignant initiation, progression and also influencing cancer cell sensitivity to chemotherapy, e.g. chemoresistance (Senthebane et al., Int. J. Mol. Sci. 2017, 18, 1586). ECM structure and composition are regulated by multiple cell types in the stroma and affect numerous aspects of tumor cell behaviour. Both genetic and non-genetic factors contribute substantially to the phenotypic diversity within tumors, but there are no approaches that can definitively resolve all their relative contributions.

The use of a synthetic polymer-based scaffold composed primarily of polyethylene glycol (PEG) modified with bioactive peptides was applied to study models of lung adenocarcinoma cell lines (Gill et al., Cancer Res; 72(22) Nov. 15, 2012). Modified PEG-RGD and MMP-sensitive hydrogels with varying elasticity and adhesive ligand concentration were applied as disks onto a glass substrate, so as to provide an array to probe and study ECM-derived differences in epithelial morphogenesis.

A biohybrid in situ-forming hydrogel (starPEG) was used to study the potential role of bone-cell-secreted factors on breast-cancer cells behaviour (Bray et al., Cancers 2018, 10, 292). The starPEG was also conjugated with matrix metalloproteinase (MMP)-cleavable peptide linkers with or without the addition of a collagen I-derived peptide to study viability, morphology, and migration of cells within their microenvironments.

Regarding the interaction between ECM proteins and drug resistance, it has been already described that ECM composition regulates drug resistance in hyaluronic acid (HA) hydrogels supplemented with fibronectin, laminin, or cyclic cell adhesion peptide RGD (cRGD) (Blehm et al., Biomaterials. 2015 July; 56: 129-139). This paper was a first evidence that the composition and architecture of the tumor-ECM environment directly affected drug efficacy, i.e. ECM features influence cancer cell sensitivity to different drugs.

Similarly, using a biomimetic hydrogel based on collagen type I with different stiffness, Lam et al. (Mol. Pharmaceutics 2014, 11, 2016-2021) already compared matrix stiffness effects on the proliferative growth and invasion of metastatic breast tumor cells and drug treatment outcomes.

Recently, an approach to screen drug responses in cells cultured on 3D biomaterial environments was developed to explore how key biophysical and biochemical features of ECM mediate drug response (Schwartz et al., Integr. Biol., 2017, 9, 912-924). A 3D PEG-maleimide (PEG-MAL) hydrogel containing cRGD was used to systematically vary stiffness, dimensionality (i.e. 2D versus 3D cultures) and cell-cell contacts to analyse matrix-mediated adaptive resistance. They identified a correlative efficacy of MEK inhibitor and sorafenib combination therapy that would not have been realized using only one screening environment, i.e. a single culture condition, or without a systems biology analysis. In said article, a single tissue type from cell lines known to be genetically homogenous was used (and not cells coming from a specific patient tumor). The need to use a selection of gels to capture the heterogeneity of cells of a specific patient tumor or of tumors of different patients is not derivable from said article.

In WO 2014/180970 A1, an array and a combinatorial method performed therewith was described. In discrete volumes of a multiwell plate, different extracellular matrix conditions were provided in an automated manner by varying the kind and/or amount of hydrogel precursor molecules, crosslinking agents, and bioactine agents attached to said hydrogel precursor molecules.

Touati et al. (Poster presentation at the AACR 2018 Annual Meeting, Chicago, Apr. 14-18, 2018) reported on the impact of different ECM compositions on the morphology of A549 lung adenocarcinoma cell line and correlated different sensitivity to drug exposure with ECM-induced cell phenotypes.

There is a need for a system that establishes ex vivo cell culture conditions for drug screening/testing that are capable to capture the different disease characteristics of a patient, in order to more accurately predict drug treatment outcomes for patients. More specifically, there is a need for a method and kit that can be readily used for assisting and improving the treatment of a patient having a certain disease.

### Preparation of Organoids

This invention refers to three-dimensional cell culture models, including any kind of cellular structures, such as organoids, tumoroids, multicellular tumor spheroids, cell spheroids, cell clusters, tumorospheres, tissue-derived tumor spheres, or fragments of the mentioned cellular structures. Hereinafter, the term “cells” is meant to refer to such any kind of cellular structures.

Organoids, including cell spheroids or clusters, are cellular three-dimensional structures of stem cells, organ-specific, tissue-specific or disease-specific cell types that develop and self-organize (or self-pattern) through cell sorting and spatially restricted lineage commitment in a manner similar to the situation in vivo. An organoid therefore represents the native physiology of the cells and has a cellular composition (including remaining stem cells and/or specialized cell or tissue types at different stages of differentiation) and anatomy that emulate the native organ, tissue and/or diseased cells and tissue situation (e.g. cancer, cystic fibrosis, Inflammatory Bowel Disease). Normal and/or diseased cells (e.g. cancer cells) can be isolated from any tissues or any cellular structures such as organoids or cancer organoids (also called tumoroids). The cells from which an organoid is generated can grow and/or differentiate to form an organ-like or disease-like tissue (e.g. cancer, cystic fibrosis, Inflammatory Bowel Disease) exhibiting multiple cell types that self-organize to form a structure very similar to the organ (i.e. cell differentiation) or diseased tissue (e.g. multicellular heterogeneity of tumors) in vivo. Organoids are therefore excellent models for studying human organs, human organ development, cancer and other diseases in a system very similar to the in vivo situation. Organoids are also used to grow and expand cells for clinical applications such as regenerative and personalized medicine.

Other examples of clinical applications are for personalized medicine where organoids representing the disease are cultured ex vivo to test drugs in order to identify personalized treatment options for the patients. Briefly, the use of patient derived cells harvested from diseased tissue biopsies or tissue resections are grown and expanded ex vivo as organoids and/or any other kind of cellular structures. Subsequently, tests with potential therapeutic treatment options (e.g. drug, combination of drugs) can be performed on these patient organoids before actually the patient is treated. Results of these ex vivo drug tests with patient cells may be used by the physicians to support their decisions on what treatment to give to the patients.

In the prior art for the above mentioned potential clinical applications, successful ex vivo patient cell growth and expansion as organoids relied on the use of an animal derived matrix (such as Matrigel®).

However, the nature of their origin, the inherent batch-to-batch variation and undefined composition of animal derived matrices such as Matrigel® prohibit regulatory approval for their use in humans or to expand cells ex vivo for subsequent transplantation in humans. In addition, these issues also may pose major obstacles for the standardization of organoid cultures that may be required for regulatory approval to use organoid drug tests for clinical diagnostics in precision medicine. As such, to translate the use of organoids to clinical applications (e.g. precision medicine, regenerative medicine, etc.), several aspects of organoid culture need to be modified. These include the development of a support matrix and of culture media that are defined and approved for human use, scalable, and preferably xeno-free (i.e. free from components of animal origin).

In Broguiere et al., Growth of Epithelial Organoids in a Defined Hydrogel, Adv. Mater. 2018, 1801621, defined but not synthetic (i.e. not allo- nor xeno-free) fibrin hydrogels supplemented with laminin-111 were shown to support the growth of organoid lines derived from the human small intestine epithelium, liver, pancreas and pancreatic ductal adenocarcinoma (PDAC).

Gjorevski (Gjorevski et al., Designer matrices for intestinal stem cell and organoid culture, Nature, Vol 539, 24 Nov. 2016, 560-56; Gjorevski et al., Synthesis and characterization of well-defined hydrogel matrices and their application to intestinal stem cell and organoid culture, Nature protocols, Vol. 12, no. 11, 2017, 2263-2274; WO 2017/036533 A1 and WO 2017/037295 A1) developed enzymatically (factor XIII) crosslinked 8-arm polyethylene glycol (PEG) hydrogels with functionalized RGD peptides and different degradation kinetics, including specific enzymatic degradations as well as controlled self-degradation kinetics (hydrolysis of PEG-acrylate) for the growth of primary mouse and human small intestinal organoids and human colorectal cancer organoids. The addition of laminin-111 purified from mouse tissue (full protein) was necessary to support organoid differentiation.

While some success of this approach was shown for the expansion and organoid formation from mouse cells, it was not shown (and rather questioned in Gjorevski 2017, p. 2265) that the above systems were suitable for the expansion and organoid formation from freshly isolated or frozen human cells from a biopsy of a human. Also, the only tested system, that is based on an enzymatic crosslinking reaction with factor XIII, is expensive, difficult to up-scale and/or to automatize for commercial purposes, and has also proven to be difficult to reproduce.

The work described by Cruz-Acuna (Cruz-Acuna et al., Synthetic hydrogels for human intestinal organoid generation and colonic wound repair, Nature cell biology, advanced online publication published online 23 Oct. 2017; DOI: 10.1038/ncb3632, 1-23; Cruz-Acuna et al., PEG-4MAL hydrogels for human organoid generation, culture, and in vivo delivery, Nature protocols, Vol. 13, september 2018, 2102-2119; and WO 2018/165565 A1) is based on developing a completely synthetic 4-arm PEG-maleimide hydrogel functionalized with RGD and crosslinked with the protease-degradable peptide GPQ-W for the growth of intestinal organoids using human embryonic stem cells and induced pluripotent stem cells. Organoids expanded in these synthetic gels were then injected into a mouse colonic injury model as a proof-of-concept study demonstrating the therapeutic potential of intestinal organoid transplantation.

It has not been shown that with this system freshly isolated or frozen cells from a biopsy of a patient could be expanded and formed into organoids. For this system, it is necessary that the crosslinker component is enzymatically degradable.

Currently, the standard for the establishment of organoid cultures ex vivo includes firstly to encapsulate freshly isolated cells (from tissues) in the “gold standard” Matrigel® (Matrigel® being one of the commercially available products of basement membrane extracts (BME)) and to grow the cells for several passages to expand them (i.e. to increase the cell number). BME (e.g. Matrigel®) is a gel derived from mouse sarcoma extract, which as already noted above has poor batch-to-batch consistency, has undefined composition and therefore cannot be used for clinical translational applications, so that obtaining regulatory approval may be challenging or impossible (Madl et al., Nature 557 (2018), 335-342).

Removing the use of gels with undefined xeno components or human components for the establishment of organoids would overcome one of the main hurdles to use organoids in clinical applications, such as regenerative medicine, precision medicine, drug testing, or patient stratifications.

Proof of concept of freshly isolated cells from biopsies cultured in fully defined (not fully synthetic) matrices has been provided by Mazzocchi et al., In vitro patient-derived 3D mesothelioma tumor organoids facilitate patient-centric therapeutic screening, Scientific reports (2018) 8:2886; Votanopoulos et al., Appendiceal Cancer Patient-Specific Tumor Organoid Model for Predicting Chemotherapy Efficacy Prior to Initiation of Treatment: A Feasibility Study, Ann Surg Oncol (2019) 26:139-147; and WO 2018/027023 A1. Briefly, cells derived from mesothelioma and appendiceal cancer patients were cultured in hyaluronic acid/collagen-based hydrogels to develop a platform for drug response prediction. However, like Matrigel® also Collagen is a naturally-derived matrix and suffers from similar problems.

So far, there has not been a report of a successful expansion of freshly isolated or frozen human cells from biopsies or tissue resections (i.e. cells which have been obtained directly from a human and which have not been pre-cultured or pre-established in another system) and subsequent formation of organoids therefrom in a fully defined and/or a fully synthetic hydrogel matrix that is not a naturally-derived matrix such as Matrigel® or collagen.

Despite the clear need for such an approach, as stated in the above discussed prior art, until now the gold standard still is the use of Matrigel® for at least the first step of expansion of the cells. This is proof for the difficulties involved in making a semi-synthetic or fully synthetic three-dimensional hydrogel system work.

There is thus also a need for providing a method for expansion of freshly isolated or frozen human cells from biopsies and subsequent formation of organoids therefrom, wherein said method completely avoids the use of a naturally-derived matrix such as Matrigel® and provides organoids suitable for clinical applications and generated in a commercially feasible manner, i.e. cost-effective, reliable, reproducible, automatizable and upscalable.

An optimal system for establishing ex vivo cell culture conditions for drug screening/testing that are capable to capture the different tumor characteristics of a patient, in order to more accurately predict drug treatment outcomes for patients, would also include the ability for expansion of freshly isolated or frozen human cells from biopsies or resections and subsequent formation of organoids therefrom, wherein said method completely avoids the use of a naturally-derived matrix such as Matrigel®.

More specifically, there is a need for a method and kit that can be readily used for assisting and improving the treatment of a patient having a certain disease, wherein said method completely avoids the use of a naturally-derived matrix such as Matrigel®.

### SUMMARY OF THE INVENTION

The present invention expands on the above discussed prior art by providing a cell growth kit that comprises extracellular matrix conditions that are specifically preselected for a certain disease or healthy tissue and thus allows a more accurate and efficient prediction of an outcome of a drug therapy for said specific disease or toxicity for said specific healthy tissue. In the above prior art, this potential of three-dimensional fully defined (including fully synthetic) hydrogels has not been recognized.

Based upon previously conducted experiments and/or knowledge, it is possible to estimate suitable conditions for growth and subsequent testing of specific tissue types, such as cancer cells or normal/healthy cells. However, while this may address certain characteristics of the respective tissue type, it is still not sufficient to address the multiple phenotypic subpopulations of a tissue type, that in e.g. the case of cancer cells vary in their ability to initiate metastases and in their sensitivity to anticancer therapy. Accordingly, even performing an assay with a specific tissue type under a single ex vivo culture condition that has been previously established as being suitable for the growth of said specific tissue type does not provide the desired optimal assistance and improvement of the treatment of a patient having a certain disease.

The present invention provides an array of extracellular matrix (ex vivo culture) conditions that are based on a preselection of extracellular matrix conditions that have been established as being suitable for a certain tissue type, but provides variations of said preselected extracellular matrix conditions. With this approach, a significantly more focused assay can be conducted. Whereas in a conventional assay with non-preselected extracellular matrix conditions (e.g. conventional screening of extracellular matrix conditions) a certain number of extracellular matrix conditions employed in said assay will not be suitable, in the method of the present invention employing preselected extracellular matrix conditions all extracellular matrix conditions are principally suitable for the intended purpose, and it is possible in a significantly more focused way to identify optimal extracellular matrix conditions for a specific phenotypic subpopulation of a certain tissue type of a patient to be treated. Thus, the present invention provides an improvement with respect to personalized medicine.

Thus, the present invention is related to a method with one tissue type, optionally in combination with other cells such as stromal cells or immune cells, comprising the steps of:
a) providing a fully defined hydrogel matrix array with discrete volumes by crosslinking, onto a substrate or into discrete volumes of a substrate, preferably a multi-well plate, different combinations of one or more different hydrogel precursor molecules, optionally in the presence of one or more biologically active molecules, optionally at least one crosslinking agent and cells of the tissue type to be tested, so as to create fully defined three-dimensional extracellular matrix conditions that differ from each other in their biological, biophysical and/or biochemical characteristics;
b) allowing said cells to grow and expand in said discrete volumes of said hydrogel matrix array in the presence of one or more different culture media;
c) performing an operation with the cells grown in said discrete volumes of said hydrogel matrix array;

wherein a specific combination of hydrogel features has been pre-selected for the said one tissue type to be tested.

According to the present invention, in step b) of the above method the cells are grown and expanded until a sufficient amount of cells is reached. If a sufficient amount of cells is reached, the desired operation (e.g. drug testing or the creation/establishment of a cell repository/biobank) can be performed in step c). Preferably, step b) (cell expansion) is performed manually, wherein increasing the cell number after each passage is important. However, it is also possible to perform step b) automatically and/or in a miniaturized manner.

Said method can be a combinatorial method, i.e. a method where a plurality of combinations of ex vivo conditions (extracellular matrix conditions, etc.) and drugs are examined simultaneously.

In one embodiment, said operation to be performed with the cells grown in said discrete volumes of said hydrogel matrix array may be the addition of one or more drugs to said discrete volumes of said hydrogel matrix array. According to said embodiment, the method is a drug screening test, in order to identify one or more drugs that are suitable for treating a condition associated with cells from the tested tissue type. This can be used in the field of personalized medicine.

If according to a preferred embodiment of the present invention said tissue type is derived from a specific patient, e.g. freshly isolated or frozen cells from a biopsy or resection of said patient, said drug screening test is an improvement as to precision and/or personalized medicine, since it helps identifying precisely the most suitable treatment for said certain patient.

According to one embodiment of the present invention, said tissue type with which the method is performed may comprise both cancer cells as well as other cell types, including stromal cells, for example cancer associated fibroblasts (CAF), or immune cells.

According to a preferred embodiment of the present invention, the tissue type is lung cancer, preferably non-small cell lung cancer overexpressing c-Met, and the hydrogel matrix is preselected as being a non self-degradable PEG hydrogel, wherein the crosslinking agent and said optional bioactive agent do not comprise any RGD motif. Preferably, the culture medium to be used in said embodiment comprises FBS (serum) or Wnt agonist such as R-spondin.

According to another preferred embodiment of the present invention, the tissue type is pancreatic ductal adenocarcinoma (PDAC) cells, and the hydrogel matrix is preselected as being a non self-degradable PEG hydrogel having a stiffness in the range of 50 to 3000 Pa, preferably 50 to 2000 Pa and most preferably 50 to 1000 Pa, wherein at least one of the crosslinking agent and/or said optional bioactive agent comprise a RGD motif. Preferably, the culture medium to be used in said embodiment comprises Wnt agonists such as R-spondin and Wnt 3a.

According to another preferred embodiment of the present invention, the tissue type is colorectal cancer (CRC) cells, and the hydrogel matrix is preselected as being PEG hydrogel having at least an initial stiffness in the range of 50 to 2000 Pa, and optionally furthermore comprising one or more biologically active molecules comprising laminin, preferably laminin-111 or laminin-511, and especially preferable natural mouse laminin-111 or recombinant human laminin-511, wherein at least one of the crosslinking agent and/or said optional bioactive agent comprise a RGD motif. Preferably, the culture medium to be used in said embodiment comprises Wnt agonists such as R-spondin and Wnt 3a.

According to another preferred embodiment of the present invention, the tissue type is breast cancer cells, and the hydrogel matrix is preselected as being preferably an enzymatic-degradable PEG hydrogel, wherein at least one of the crosslinking agent preferably comprises an enzymatically degradable motif, preferably a MMP-sensitive motif and said hydrogel optionally furthermore comprises one or more biologically active molecules comprising laminin, preferably laminin-111, and especially preferable natural mouse laminin-111. Preferably, the culture medium to be used in said embodiment comprises FBS (serum) or Wnt agonist such as R-spondin.

According to another preferred embodiment of the present invention, the tissue type is cancer cells that grow ex vivo more slowly than their healthy/normal counterparts (e.g. epithelial and/or stromal cells), preferably prostate cancer cells, and the hydrogel matrix is preselected as being a PEG hydrogel, preferably having a stiffness in the range of 50 to 2000 Pa, wherein said crosslinking agent and said optional bioactive agent do not comprise any RGD motif.

According to another preferred embodiment of the present invention, the tissue type is cancer cells, preferably pancreatic ductal adenocarcinoma (PDAC) cells, in combination with stromal cells, preferably fibroblasts, and the hydrogel matrix is preselected as being a PEG hydrogel having a stiffness preferably in the range of 50 to 3000 Pa, preferably 50 to 2000 Pa and most preferably 50 to 1000 Pa, wherein at least one of the crosslinking agents comprises an enzymatically degradable motif, preferably a MMP-sensitive motif, and wherein at least one of the crosslinking agent and/or said optional bioactive agent comprise a RGD motif. Preferably, the culture medium to be used in said embodiment comprises Wnt agonists such as R-spondin and Wnt 3a, and more preferably also FBS (fetal bovine serum).

In another embodiment, said operation to be performed with the cells grown in said discrete volumes of said hydrogel matrix array may be drug screening/testing on healthy organoid cells, in particular in the field of precision medicine. According to a preferred embodiment, healthy/normal organoids (e.g. colon or intestinal organoids, normal/healthy prostate cells or healthy cells of other organs) may be used as control conditions and/or as cytotoxic assay (e.g. to test the toxicity of a drug) in drug tests with diseased cells of the same organ. For example, healthy/normal colon or intestinal organoids can be used in drug tests as control conditions where drugs are tested on, e.g. cancer organoids or organoids from cystic fibrosis tissues of the same patient.

In another embodiment, said operation to be performed with the cells grown in said discrete volumes of said hydrogel matrix array may be the isolation of the grown cells (designated herein as organoids) in order to use said 3D cellular structures in basic scientific research or to implant said cells into a human, for the purposes of regenerative or personalized medicine.

A significant advantage of a preferred embodiment of the method of the present invention is that the use of a naturally-derived matrix such as Matrigel® can be completely avoided. It has been surprisingly found that this long-felt need in the art can be achieved by using specifically pre-selected conditions as described hereinafter. Performing the entire method under fully-defined extracellular matrix conditions provides more precise results for drug screening, since any varying behaviour of a drug can be clearly attributed to a specific extracellular matrix condition. Also, performing the entire method under fully-defined extracellular matrix conditions meets the regulatory approval requirements for personalized and regenerative medicine, in contrast to the methods performed in the prior art.

The present invention is also related to a kit of parts for performing an operation on or with one or more tissue type, comprising:
a) components for preparing a fully defined hydrogel matrix array, so as to create fully defined three-dimensional extracellular matrix conditions that differ from each other in their biological, biophysical and/or biochemical characteristics, said components comprising
   one or more different hydrogel precursor molecules,
   optionally at least one crosslinking agent,
   optionally one or more biologically active molecules,
b) one or more different culture media,

wherein a specific combination of hydrogel features has been pre-selected for the tissue type to be tested.

According to a preferred embodiment of the present invention, said kit is for testing the influence of drugs on lung cancer cells, preferably non-small cell lung cancer cells, overexpressing c-Met, the hydrogel matrix is preselected as being a non self-degradable PEG hydrogel, wherein the crosslinking agent and said optional bioactive agent do not comprise any RGD motif, and said culture medium preferably comprises FBS (serum) or a Wnt agonist such as R-spondin.

According to another preferred embodiment of the present invention, said kit is for testing the influence of drugs on pancreatic ductal adenocarcinoma (PDAC) cells, the hydrogel matrix is preselected as being a non self-degradable PEG hydrogel having a stiffness in the range of 50 to 3000 Pa, preferably 50 to 2000 Pa and most preferably 50 to 1000 Pa, wherein at least one of the crosslinking agent and/or said optional bioactive agent comprise a RGD motif, and said culture medium preferably comprises Wnt agonists such R-spondin and Wnt 3a.

According to another preferred embodiment of the present invention, said kit is for testing the influence of drugs on colorectal cancer (CRC) cells, and the hydrogel matrix is preselected as being PEG hydrogel having at least an initial stiffness in the range of 50 to 2000 Pa, and optionally furthermore comprising one or more biologically active molecules comprising laminin, preferably laminin-111 or laminin-511, and especially preferable natural mouse laminin-111 or recombinant human laminin-511, wherein at least one of the crosslinking agent and/or said optional bioactive agent comprise a RGD motif, and wherein said culture medium preferably comprises Wnt agonists such as R-spondin and Wnt 3a.

According to another preferred embodiment of the present invention, said kit is for testing the influence of drugs on breast cancer cells, and the hydrogel matrix is preselected as being preferably an enzymatic-degradable PEG hydrogel, wherein at least one of the crosslinking agent preferably comprises an enzymatically degradable motif, preferably a MMP-sensitive motif, and said hydrogel optionally furthermore comprises one or more biologically active molecules comprising laminin, preferably laminin-111, and especially preferable natural mouse laminin-111, and wherein said culture medium preferably comprises FBS (serum) or Wnt agonist such as R-spondin.

According to another preferred embodiment of the present invention, said kit is for growing and testing the influence of drugs on cancer cells that grow ex vivo more slowly than their healthy/normal counterparts (e.g. epithelial and/or stromal cells), preferably prostate cancer cells. The hydrogel matrix is preselected as being a PEG hydrogel, preferably having a stiffness in the range of 50 to 2000 Pa, wherein said crosslinking agent and said optional bioactive agent do not comprise any RGD motif.

The kits according to the present invention are designated for cells coming from a specific tissue type and can be readily used for performing operations on or with said tissue type, such as testing the influence of drugs on said tissue type, or the isolation of the grown cells in order to use said grown cells in basic scientific research, personalized medicine or to implant said cells into a human, for the purposes of regenerative medicine, or for drug development/discovery or the creation of a cell repository/biobank. The kits according to the present invention are correspondingly indicated, e.g. by instructions provided with said kit, for the said specific tissue type with which it is to be used.

### DETAILED DESCRIPTION OF THE INVENTION

### Definitions

This invention refers to three-dimensional cell culture models, including any kind of cellular structures, such as single cells, organoids, tumoroids, multicellular tumor spheroids, cell spheroids, cell clusters, tumorospheres, tissue-derived tumor spheres, or fragments of the mentioned cellular structures.

Hereinafter, the term “cells” is meant to refer to such any kind of cellular structures.

An array is a set of several discrete volumes that can be arranged in a certain manner, for example in rows and/or columns. For example, a typically used well plate (e.g. a 48-well plate) provides 48 discrete volumes that are arranged in 8 columns and 6 rows, wherein in this example each column consist of 6 discrete volumes. Each such column in this example is considered as an array, according to the present invention. Alternatively, in this example also each row consisting of 8 discrete volumes can be considered as an array.

Organoids, including cell spheroids or clusters, are cellular three-dimensional structures of stem cells, organ-specific, tissue-specific or disease-specific cell types that develop and self-organize (or self-pattern) through cell sorting and spatially restricted lineage commitment in a manner similar to the situation in vivo. An organoid therefore represents the native physiology of the cells and has a cellular composition (including remaining stem cells and/or specialized cell or tissue types at different stages of differentiation) and anatomy that emulate the native organ, tissue and/or diseased cells and tissue situation (e.g. cancer, cystic fibrosis, Inflammatory Bowel Disease). Normal and/or diseased cells (e.g. cancer cells) can be isolated from any tissues or any cellular structures such as organoids or cancer organoids (also called tumoroids). The cells from which an organoid is generated can grow and/or differentiate to form an organ-like or disease-like tissue (e.g. cancer, cystic fibrosis, Inflammatory Bowel Disease) exhibiting multiple cell types that self-organize to form a structure very similar to the organ (i.e. cell differentiation) or diseased tissue (e.g. multicellular heterogeneity of tumors) in vivo. Organoids are therefore excellent models for studying human organs, human organ development, cancer and other diseases in a system very similar to the in vivo situation. Organoids are also used to grow and expand cells for clinical applications such as regenerative and personalized medicine.

According to the present invention, the term “tissue type” refers to a group of cells that have a similar structure and act together to perform a specific function. In animals, there are four different tissue types: connective, muscle, nervous, and epithelial tissue. According to the present invention, cells from the same tissue type are an ensemble of cells that act together to carry out a specific function, when being healthy cells. More preferably, according to the present invention cells of the same tissue type have the same origin in the human body (e.g. breast cells).

According to the present invention, it is to be understood that the same tissue type encompasses both healthy (or also called normal) and diseased cells, such as cancer cells. Cells from the same tissue type may contain different cell types/subtypes, such as different cell populations (e.g. multicellular heterogeneity of tumors).

According to a preferred embodiment of the present invention, said tissue type with which the method of the invention is performed may comprise both cancer cells as well as other cell types, including stromal cells, for example cancer associated fibroblasts (CAF), or immune cells.

Examples of tissue types to be used for the purposes of the present invention are lung cancer, preferably non-small cell lung cancer, overexpressing c-Met; pancreatic ductal adenocarcinoma (PDAC) cells (preferably in combination with stromal cells, preferably fibroblasts), colorectal cancer (CRC) cells, breast cancer cells, or cancer cells that grow ex vivo more slowly than their healthy/normal counterparts (e.g. epithelial and/or stromal cells), preferably prostate cancer cells.

According to the present invention, the term “freshly isolated or frozen human cells from biopsies or tissue resections” refers to cells which have been obtained directly from a human by any of the mentioned procedures and which have not been pre-cultured or pre-established in another system before being used in a method of forming organoids, spheroids, cell clusters or any cellular structures. Typically, such fresh cells are collected and used in the method of the present invention immediately or within a period of up to 3 to 4 days. If the cells are not used immediately after collection, they may be frozen for storage purposes, under conventionally used conditions. The collected cells may be single and/or “clusters” of cells, including dissociated cells, crypts and pieces of tissue. According to a preferred embodiment of the present invention, epithelial cells are used.

According to the present invention, the term “de novo formation of organoids” refers to freshly isolated or frozen human cells (e.g. human biopsy or tissue resection) that have been grown ex-vivo (i.e. outside the original organism) for the first time. The terms “First ex-vivo cell growth” or “Passage zero (PO)” can be used synonymously.

According to the present invention, the term “Pre-established organoids” refers to cells, single cells and/or cell clusters (e.g. cell aggregates, organoids, etc.) that have been grown in other systems (e.g. Matrigel®, 2D or 3D systems, in vivo as patient-derived xenografts (PDX)) before being applied to the hydrogel of the present invention.

According to the present invention, the term “cell growth” refers to the successful growth of cells.

According to the present invention, the term “Cell passaging” or “passage” or “cell splitting” or “organoid passaging” refers to the steps of extracting cells from one gel and seeding and growing those cells in another gel having the same or different characteristics as/than the previous gel.

According to the present invention, the term “Cell expansion” or “organoid expansion” refers to the steps of cell growth and cell number increase (e.g. within the same passage or from one passage to the next one).

According to the present invention, the term “Organoid differentiation” refers to the successful induction of cell differentiation in an organoid.

According to the present invention the term “fully defined hydrogel” refers to a hydrogel selected form the group consisting of fully synthetic or semi synthetic hydrogels, i.e. a hydrogel that has a fully defined structure and/or composition, due to the known nature of the precursor molecules used for its synthesis and its route of synthesis.

According to the present invention the term “fully synthetic hydrogel” refers to a hydrogel that has been formed exclusively from synthetic precursors, i.e. in the absence of any naturally derived precursor such as natural laminin-111.

According to the present invention the term “fully defined semisynthetic hydrogel” refers to a hydrogel that comprises at least one naturally derived precursor such as natural laminin-111, but has a fully defined structure and/or composition, due to the known nature of the precursor molecules used for its synthesis. A fully defined semi-synthetic hydrogel thus differs from naturally-derived hydrogels such as Matrigel®, which have an unknown structure and/or composition.

According to the present invention, the term “encapsulated in a cell culture microenvironment” or similar expressions mean that the cell(s) is/are embedded in a matrix in such a way that they are completely surrounded by said matrix, thereby mimicking naturally occurring cell growth conditions.

As understood herein, the term “microenvironment” or “volume of microenvironment”, respectively, means a volume that is suitable for high-throughput testing appliances, in particular multi-well plates. Typical volumes being analysed in multi-well plates are in the range of about 100 nl to about 500 μl, preferably of about 2 μl to about 50 μl.

The term “discrete volumes” relates to spatially separated spots or areas within the array. The separated spots or areas may be in contact with each other or preferably separated from each other, e.g. by a plastic barrier. Into or onto each of these discrete volumes, cells of a desired tissue type can be placed in such a way that they are separated from each other. They do not come into contact with each other from the beginning of an experiment and remain so over time, thereby growing independently from a neighbouring volume and only under the influence of their cell culture microenvironment (ex vivo culture).

The term “crosslinking agent” refers to a chemical substance that comprises at least two functional groups that are capable of reacting with functional moieties of hydrogel precursor molecules, so as to link two or more hydrogel precursor moieties with each other. Examples are peptides comprising at least two functional groups such as cysteine moieties, or a polyethylene glycol having at least two functional groups such as thiol groups (e.g. a 2-arm or multi-arm PEG with terminal thiol moieties).

The term “crosslinkable by cell-compatible reaction(s)” (or similar terminology), comprises reactions both on the basis of (i) covalent bond formation, chosen from the group consisting of a) enzymatically catalysed reactions, preferably depending on activated transglutaminase factor XIIIa; and b) not-enzymatically catalysed and/or uncatalysed reactions, preferably a Michael addition reaction; and/or ii) non-covalent bond formation (e.g. on the basis of hydrophobic interactions, H-bonds, van-der-Waals or electrostatic interactions; in particular induced by temperature changes or changes in ionic strength of a buffer). These reactions can take place between two hydrogel precursor molecules comprising functional groups that may react with each other, or between at least one hydrogel precursor molecule and a crosslinking agent which comprise functional groups that may react with each other.

According to the present invention, the term “performing an operation with the cells grown” includes the addition of one or more drugs to said discrete volumes of said hydrogel matrix array. Thus, the method may be for drug development or a drug screening test, in order to identify one or more drugs that are suitable for treating a condition associated with cells from the tested tissue type. This can be used in the field of personalized medicine. The method can also be used for drug discovery, as a cytotoxicity assay, or in regenerative medicine. The term “performing an operation with the cells grown” includes also operations where the cells themselves or by-products from the cells are analysed, e.g. with DNA or RNA sequencing methods such as NGS (next generation sequencing), as well as operations where products of these cells are analysed (e.g. supernatant analysis) or cell-derived products (such as the products of cell passaging or cell expansion) are isolated for further use (e.g. for establishment of a biobank or cell repository, or for the establishment of organoids).

According to the present invention, the term “pre-selected” means that the extracellular matrix conditions, i.e. at least one of said hydrogel precursor molecules, said optional crosslinking agent, said optional bioactive agent, and preferably said culture media, preferably at least two of them and most preferably all of them, are selected for the tissue type to be tested such that a specific combination of hydrogel features has been pre-selected. Hydrogel features are features that define the structure and/or function of a hydrogel. Examples are the chemical structure of the hydrogel (as governed by the precursor and optional crosslinking agents and optional bioactive agents employed), the stiffness or the degradation properties (e.g. by hydrolysis or enzymatic reaction) of the hydrogel. As compared to the discussed prior art methods, according to the present invention the extracellular matrix conditions are not chosen randomly. Based on previously obtained or available information, extracellular matrix conditions are chosen that are already known to be suitable for the growth and manifestation of a phenotypic characteristic of interest of the specific tissue type to be tested. Methods of pre-selecting extracellular matrix conditions will be described below. According to the present invention, also the variations of pre-selected extracellular matrix conditions employed in the method of the invention are to be understood as “pre-selected”, since these variations are not random, but based on the preselected extracellular matrix conditions.

According to the present invention, the term “variations of preselected extracellular matrix (ex vivo culture) conditions” encompasses conditions that are similar to the preselected conditions, but differ in at least one parameter, preferably 1 to 3 parameters, such as hydrogel features (e.g. stiffness, degradation), components of a culture medium, amount of a component in the extracellular matrix conditions, bioactive agents in the extracellular matrix, etc. Generally, the differing parameters are biological (e.g. presence or absence of a RGD motif), biophysical (e.g. stiffness of the hydrogel) and/or biochemical characteristics (e.g. enzymatic degradation).

According to the present invention, the term “self-degradable” means that the hydrogel degrades over time without the influence of a degrading enzyme. Preferably, self-degradation occurs due to hydrolysis of bonds in the hydrogel which are susceptible to reaction with water. As an example, ester bonds formed by the reaction of acrylate groups in PEG-Acr precursor molecules (i.e. precursor molecules containing a PEG molecule with terminal acrylate groups) may be mentioned.

According to the present invention, the term “non self-degradable” means that the hydrogel does not degrade over time without the influence of a degrading enzyme. Non self-degradable hydrogels do not comprise bonds in the hydrogel which are susceptible to reaction with water. As an example, hydrogels formed from PEG-VS precursor molecules (i.e. precursor molecules containing a PEG molecule with terminal vinylsulfone groups) may be mentioned.

According to the present invention, the term “RGD” or “RGD sequence” refers to a minimal bioactive RGD sequence, which is the Arginine-Glycine-Aspartic Acid (RGD) sequence, and which is the smallest (minimal) fibronectin-derived amino acid sequence that is sufficient to mimic cell binding to fibronectin and/or to promote adhesion of the anchorage-dependent cells. Moreover, lysine- or arginine-containing amino acid sequences, such as RGD, are suitable substrates for proteases such as trypsin-like enzymes used e.g. for gel dissociation. Examples of suitable RGD motifs are RGD, RGDS, RGDSP, RGDSPG, RGDSPK, RGDTP, RGDSPASSKP, PHSRNSGSGSGSGSGRGDSPG or any cyclic RGD motifs such as cyclo (RGDfC), but principally any known and successfully employed RGD sequences, in the field of hydrogels and cell culture, could be used.

The shear modulus of a hydrogel is equivalent to the modulus of rigidity, G, elastic modulus or elasticity of a hydrogel. The shear modulus is defined as the ratio of shear stress to the shear strain. The shear modulus of a hydrogel can be measured using a rheometer. In brief, preformed hydrogel discs 1-1.4 mm in thickness are allowed to swell in complete cell culture medium for at least 3 h, and are subsequently sandwiched between the parallel plates of the rheometer. The mechanical response of the gels is recorded by performing frequency sweep (0.1-10 Hz) measurements in a constant strain (0.05) mode, at room temperature. The shear modulus (G′) is reported as a measure of gel mechanical properties.

### Method of Making the Hydrogel Matrix Array

A hydrogel matrix array according to the present invention can be generally made as described in WO 2014/180970 A1.

Briefly, said preferred method comprises the steps of
a) providing one or more different hydrogel precursor molecules, and optionally at least one crosslinking agent;
b) combining and dispensing different combinations of hydrogel precursor molecules according to step a), and optionally at least one crosslinking agent, onto a substrate or into discrete volumes of a substrate, preferably a multi-well plate in an automated manner;
c) adding to said discrete volumes one or more biologically active molecules and either attaching said molecules to at least one of the hydrogel precursor molecules present or the hydrogel formed in step e) or allowing them to diffuse freely;
d) adding cells onto/into said discrete volumes of the substrate; and
e) crosslinking said hydrogel precursor molecules to form a hydrogel matrix by cell-compatible crosslinking reactions, such as an enzymatically catalysed reaction, or a Michael addition reaction.

The hydrogels used, which are obtained by cross-linking hydrogel precursor molecules, can be principally selected from any type of synthetic or semi-synthetic well defined hydrogels known in the art. Examples are photo-crosslinkable hydrogels, such as the hydrogels which are made using a reaction mechanism via a radically mediated thiol-norbornene (thiol-ene) photopolymerization to form hydrogels (Anseth et al., Adv Mater. 2009 Dec. 28; 21(48): 5005-5010; Nature scientific reports 2015, 5:17814), or hydrogels that are prepared by click-chemistry (such as Michael addition reaction), physical crosslinking, or enzymatic crosslinking.

The hydrogels used, which are obtained by cross-linking hydrogel precursor molecules, are preferably hydrophilic polymers such as poly(ethylene glycol) (PEG)-based polymers, most preferably multiarm PEG-based polymers that are crosslinked by cell-compatible crosslinking reactions. The specific hydrogels to be used depend on the results of pre-selection for a specific tissue type and will be discussed in the preferred embodiments below.

Preferably, PEG-based hydrogels are used that are composed of PEG (polyethylene glycol) precursor molecules that are crosslinkable using either thrombin-activated Factor XIIIa under physiological conditions by a crosslinking mechanism that is detailed in Ehrbar et al. (Ehrbar, M., Rizzi, S. C., Schoenmakers, R. G., Miguel, B. S., Hubbell, J. A., Weber, F. E., and Lutolf, M. P., Biomolecular hydrogels formed and degraded via site-specific enzymatic reactions, Biomacromolecules 8 (2007), 3000-3007), or via mild chemical reactions by a crosslinking mechanism as e.g. detailed in Lutolf et al. (Lutolf, M. P., and Hubbell, J. A., Synthesis and physicochemical characterization of end-linked poly(ethylene glycol)-co-peptide hydrogels formed by Michael-type addition, Biomacromolecules 4, 713-722).

A preferred hydrogel of the present invention is based on a multi-arm PEG (poly(ethylene glycol)) containing ethylenically unsaturated groups selected from the group consisting of vinylsulfone or/and acrylate moieties, as a precursor molecule.

According to a preferred embodiment of the present invention, the multi-arm PEG is selected from the group consisting of PEG bearing 2 to 12 arms, preferably 4-arms or 8-arms, i.e. preferably is a 4-arm or 8-arm PEG. The PEG can have a molecular weight from 1,000-1,000,000, from 1,000-500,000, from 1,000-250,000, from 1,000-150,000, from 1,000-100,000, from 1,000-50,000, from 5,000-100,000, from 5,000-50,000, from 10,000-100,000, from 10,000-50,000, from 20,000-100,000, from 20,000-80,000, from 20,000-60,000, from 20,000-40,000, or from 40,000-60,000. The above molecular weights are average molecular weights in Da., as determined by e.g. methods such as GPC or MALDI.

Such PEGs are known in the art and commercially available. They consist of a core that in case of a 4-arm PEG may be pentaerythritol, and in the case of an 8-arm PEG may be tripentaerythritol or hexaglycerol:

| | |
|---|---|
| 4-arm PEG | 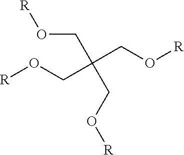 |
| 8-arm PEG | 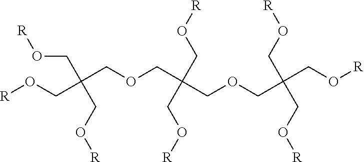 |
| 8-arm PEG | 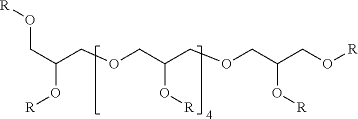 |

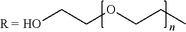

In a 4-arm PEG-VS or 8-arm PEG-VS, the terminal free OH groups of the above 4-arm PEG or 8-arm PEG are converted under conditions known in the art into vinylsulfone groups, so that in the above formulas R becomes for example

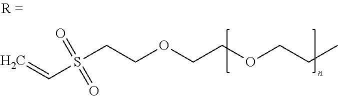

In a 4-arm PEG-Acr or 8-arm PEG-Acr, the terminal free OH groups of the above 4-arm PEG or 8-arm PEG are converted under conditions known in the art into acrylate groups, so that in the above formulas R becomes for example

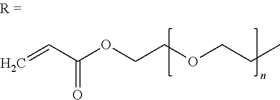

Preferably, all terminal free OH groups of the above 4-arm PEG or 8-arm PEG are converted into vinylsulfone or acrylate moieties.

Vinylsulfone or acrylate moieties are ethylenically unsaturated groups that are suitable for crosslinking the PEG precursor molecules via a Michael addition reaction. The Michael addition reaction is a well-known chemical reaction that involves the reaction of a suitable nucleophilic moiety with a suitable electrophilic moiety. It is known that, for example, acrylate or vinylsulfone moieties are suitable Michael acceptors (i.e. electrophiles) that react with e.g. thiol moieties as suitable Michael donors (i.e. nucleophiles).

A hydrogel (gel) is a matrix comprising a network of hydrophilic polymer chains. A biofunctional hydrogel is a hydrogel that contains bio-adhesive (or bioactive) molecules, and/or cell signalling molecules that interact with living cells to promote cell viability and a desired cellular phenotype.

For obtaining the hydrogel according to a preferred embodiment of the present invention, the above PEG precursor molecule is accordingly reacted with a crosslinker molecule containing at least two, preferably two nucleophilic groups capable of reacting with said ethylenically unsaturated groups of said multi-arm PEG in a Michael addition reaction. A crosslinker molecule is a molecule that connects at least two of the above PEG precursor molecules with each other. For that purpose, the crosslinker molecule has to possess at least two, preferably two of the above nucleophilic groups, so that one nucleophilic group reacts with the first PEG precursor molecule and the other nucleophilic group reacts with a second PEG precursor molecule. According to a preferred embodiment of the present invention, said crosslinker molecule is a peptide comprising at least two RGD motifs and at least two cysteine moieties. Cysteine is an amino acid that comprises a thiol group, i.e. a Michael donor moiety.

Cross-linking of the hydrogel precursor molecules is done in the presence of tissue types to be studied in discrete volumes of the array, in such a way that the cells are encapsulated by the hydrogel matrix, i.e. are residing in a distinct cell culture microenvironment.

Mechanical properties of the three-dimensional hydrogel matrix according to the invention can be changed by varying the polymer content of the cell culture microenvironments, as well as the molecular weight and/or functionality (number of sites available for crosslinking) of the polymeric gel precursors. Thus, e.g. the stiffness of the matrix, represented by shear modulus (G′), can vary between 10 to 10000 Pa, preferably 50 to 1000 Pa for soft gels or 1000-2000 Pa for medium gels or 2000-3000 Pa for hard gels. The shear modulus of a hydrogel is equivalent to the modulus of rigidity, G, elastic modulus or elasticity of a hydrogel. The shear modulus is defined as the ratio of shear stress to the shear strain. The shear modulus of a hydrogel can be measured using a rheometer. In brief, preformed hydrogel discs 1-1.4 mm in thickness are allowed to swell in aqueous solution (e.g. in a buffer or in complete cell culture medium) for at least 3 h, and are subsequently sandwiched between the parallel plates of the rheometer. The mechanical response of the gels is recorded by performing frequency sweep (0.1-10 Hz) measurements in a constant strain (0.05) mode, at room temperature. The shear modulus (G′) is reported as a measure of gel mechanical properties.

Further, physicochemical properties of the matrix over time can be changed by conferring degradation characteristics to the gel matrix via incorporation into the matrix of peptides of different sensitivities to cell-secreted proteases such as matrix-metalloproteinases (MMPs), plasmin or cathepsin K. This renders the hydrogel matrix “enzymatically degradable”. Susceptibility to proteases and the resulting change in physicochemical properties of the matrix when proteases are secreted by the cells allows for efficient cell proliferation and migration in the three-dimensional matrix. To match the mechanical properties of hydrogel matrices having different susceptibilities to proteolytic degradation, the precursor content of the matrix can be fine-tuned by varying the polymer precursor content of the matrix, the molecular weight and/or functionality (number of sites available for crosslinking) of the polymeric gel precursors. The desired stiffness range is achieved by fixing the sum of the polymer (PEG) content and the crosslinker content within the hydrogel accordingly, preferably to 1.0-10% w/v. In a PEG-based hydrogel matrix, susceptibility to proteases can be changed e.g. by incorporating different peptide sequences with different sensitivities to cell-secreted proteases into the matrix precursor molecules.

Biological properties of cell culture microenvironments can be modulated by addition of one or more biologically active molecules to the matrix. As used herein, these biologically active molecules may be selected e.g. from the group of
i) extracellular matrix-derived (ex vivo culture) factors;
ii) cell-cell interaction factors; and/or
iii) cell signalling factors.

The extracellular matrix-derived factors i) used may be, for instance, ECM proteins such as laminins, collagens, elastins, fibronectin or elastin, proteoglycans such as heparin sulfates or chondroitin sulfates, non-proteoglycan polysaccharides such as hyaluronic acids, or matricellular proteins such as fibulins, osteopontin, periostin, SPARC family members, tenascins, or thrombospondins. These ECM factors can either be used in a full-length version or as smaller, functional building blocks such as peptides and oligosaccharides, or glycosaminoglycans such as hyaluronic acid (also called hyaluronan).

The cell-cell interaction proteins ii), mostly transmembrane proteins, used may be proteins involved in cell-cell adhesion such as cadherins, selectins or cell adhesion molecules (CAMs) belonging to the Ig superfamily (ICAMs and VCAMs) or components of transmembrane cell signalling system such as Notch ligands Delta-like and Jagged.

The cell signalling factors iii) used may be growth factors or developmental morphogens such as those of the following families: adrenomedullin (AM), angiopoietin (Ang), autocrine motility factor, bone morphogenetic proteins (BMPs), brain-derived neurotrophic factor (BDNF), epidermal growth factor (EGF), Erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), leukaemia inhibitory factor (LIF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β) tumor-necrosis-factor-alpha (TNF-α), vascular endothelial growth factor (VEGF), Wnt signalling pathway, placental growth factor (PlGF), or members of the large class of cytokines or chemokines.

Extracellular matrix-derived i) and cell-cell interaction factors ii) can be site-specifically attached to the hydrogel matrix either before or during cross-linking. Gel functionalization with biologically active molecules can be achieved by direct covalent bond formation between free functional groups on the biomolecule (e.g. amine or thiol groups) or a peptidic substrate for a crosslinking enzyme (e.g. a transglutaminase) and the gel network, or via affinity binding between a domain on a chimeric/tagged protein and an auxiliary protein attached to the gel. The tagged proteins include those having Fc-tags, biotin-tags or His-tags such as to enable binding to ProteinA (or ProteinG, ProteinA/G), Streptavidin (or NeutrAvidin) or NTA.

Alternatively, those factors may be part of the crosslinking agent, and by virtue of the crosslinking reaction described herein may be incorporated into the hydrogel polymer.

The biomolecules may require different gel-tethering strategies to the hydrogel networks. Larger ex vivo culture-derived or ex vivo culture-mimetic proteins and peptides are preferably attached to the hydrogel by non-specific tethering using linear, heterodifunctional linkers. One functional group of this linker is reactive to the functional groups attached to termini of the polymer chains, preferably thiols. The other functional group of the linker is capable of non-specifically tethering to the biomolecule of interest via its amine groups. The latter functional group is selected from the group consisting of succinimidyl active ester such as N-hydroxysuccinimide (NHS), succinimidyl alpha-methylbutanoate, succinimidyl propionate; aldehyde; thiol; thiol-selective group such as acrylate, maleimide or vinylsulfone; pyridylthioesters and pyridyldisulfide. Preferably NHS-PEG-maleimide linkers are attached to the biomolecules.

The cell signalling factors iii) can either be added to the crosslinked hydrogel matrix encapsulating cells in a soluble form in spatially separate areas and thus are allowed to diffuse freely into the matrix to reach the cells. Alternatively, they can be tethered to the matrix in the same way as described above for extracellular matrix-derived i) and cell-cell interaction factors ii).

Step b) of the above described preferred method is carried out using an automated method for gel fabrication and miniaturized samples in order to achieve the required level of diversity in formulating 3D cell-containing matrices having large numbers of different cell culture microenvironments and to also achieve the required repetitions. To this end, a commercially available liquid handling robot is preferably used to accurately synthesize volumes as low as 100 to 500 nanoliters of each of the unique mixture of precursor molecules according to step a) preferably in triplicate, in a completely automated manner, onto a substrate, such as a glass slide or, preferably, into a multi-well plate such as a standard 1536-well plate. The latter format is preferred as it presents an ideal surface to volume ratio for the selected hydrogel drops and represents a standard format which can be adapted to various experimental setups. Once the 3D hydrogel matrix is generated, the system can function as a multimodal assay platform, where multiple readouts can be obtained in parallel.

According to another preferred embodiment, the components making up the final hydrogel are lyophilized and provided as an unreacted powder, which is re-solubilized manually or automatically, using a handling robot, with an appropriate buffer to form a hydrogel. The desired cell suspension is added before gelation occurs, and the 3D hydrogel matrix is generated as above.

In step e) the cross-linking of the hydrogel precursor molecules to form a three-dimensional hydrogel matrix can be achieved by using at least one cross-linking agent. When PEG-based precursor molecules are used, for example a chemically reactive bi-functional peptide can be chosen as cross-linking agent. An example are the mild chemical reactions by a crosslinking mechanism as detailed in Lutolf et al. (Lutolf, M. P., and Hubbell, J. A., Synthesis and physicochemical characterization of end-linked poly(ethylene glycol)-co-peptide hydrogels formed by Michael-type addition, Biomacromolecules 4, 713-722 (2003)). However, it is also conceivable that the crosslinking may occur immediately upon combination of two different precursor molecules which are readily reactive towards each other (such as e.g. by highly selective so-called click chemistry such as e.g. the Michael-type addition reaction or other chemical reactions).

It is also conceivable that the crosslinking may occur upon combination of two different precursor molecules which are reactive towards each other, or of one type of precursor molecule having different kinds of moieties which are reactive towards each other, in the presence of a catalyst such as an enzyme. An example are PEG (polyethylene glycol) precursor molecules that are cross-linkable using thrombin-activated Factor XIIIa under physiological conditions by a crosslinking mechanism that is detailed in Ehrbar et al. (Ehrbar, M., Rizzi, S. C., Schoenmakers, R. G., Miguel, B. S., Hubbell, J. A., Weber, F. E., and Lutolf, M. P., Biomolecular hydrogels formed and degraded via site-specific enzymatic reactions, Biomacromolecules 8 (2007), 3000-3007). Briefly, to create suitable hydrogel precursors, 8-arm PEG-VS and/or 8-arm PEG-Acr macromers are end-functionalized with lysine- and glutamine-presenting peptides that serve as substrates for the activated transglutaminase factor XIII (FXIIIa). The crosslinking of the macromers and resulting gel formation occurred through the FXIIIa-mediated formation of ε-(α-glutamyl)lysine isopeptide side-chain bridges between the two peptide substrates.

The array of dispensed hydrogel precursors can be stored and used (i.e. brought in contact with cells for screening experiments) at a later time point. Storage is preferably conducted in a multi well plate (e.g. 96-, 384- or 1536-well plate) and can either be done using precursors in solution (with yet a crosslinking agent missing) or else lyophilized precursors, i.e. a powder. The powder is and remains unreacted. Upon e.g. addition of a buffer, the lyophilized precursors are solubilised and may then react with each other.

### Pre-Selection

According to the present invention, the ex vivo conditions (e.g. extracellular matrix conditions) to be employed are pre-selected for the tissue type to be tested.

According to one embodiment of the present invention, said preselection can be carried out by a method as described in WO 2014/180970 A1.

In more detail, according to a preferred embodiment the cells from a specific tissue type to be used are subjected to a method with randomly chosen ex vivo conditions, comprising the steps:
a) providing one or more different hydrogel precursor molecules, and optionally at least one crosslinking agent, for building up cell culture microenvironments;
b) combining and dispensing different combinations of hydrogel precursor molecules, and optionally at least one crosslinking agent, according to step a) onto a substrate or into discrete volumes of a substrate, preferably a multi-well plate, preferably in an automated manner;
c) further adding to said discrete volumes of said substrate one or more biologically active molecules and either attaching said molecules to at least one of the hydrogel precursor molecules present or the hydrogel formed in step e) or allowing them to diffuse freely;
d) adding cells of the specific tissue type onto/into said discrete volumes of the substrate;
e) crosslinking said hydrogel precursor molecules by cell-compatible crosslinking reactions, such as an enzymatically catalysed reaction, or a Michael addition reaction, to form a hydrogel matrix;
f) allowing said cells of the specific tissue type to grow in said discrete volumes of said hydrogel matrix;
g) monitoring said cells of the specific tissue type during step f) over time;
h) determining the behaviour for different cell culture microenvironments;
i) identifying a specific cell culture microenvironment or range of cell culture microenvironments that provides suitable conditions for growth of the different cell populations from said specific tissue type.

The specific cell culture microenvironment or range of cell culture microenvironments identified in step i) above are used as pre-selected extracellular matrix conditions for the method of the present invention.

As described herein, in the method of the present invention a hydrogel matrix array is provided with said pre-selected extracellular matrix conditions and variations of said pre-selected extracellular matrix conditions. As compared to the method described in WO 2014/180970 A1, by using pre-selected extracellular matrix conditions and variations of said pre-selected extracellular matrix conditions, a more focused and precise assay can be conducted, that allows for the identification of specific treatment methods and/or for different behaviour of e.g. multiple phenotypic and/or genotypic subpopulations of a tissue type.

If from the prior art suitable ex vivo conditions (e.g. ECM conditions) are already known, a method as described in WO 2014/180970 A1 does not have to be conducted, but instead the known suitable ex vivo conditions (e.g. extracellular matrix conditions) can be directly employed in the method and kit of the present invention.

### Kit of Parts

According to one aspect of the present invention, kits of parts can be provided which comprise the preselected extracellular matrix conditions for one specific tissue type. Such a kit of part can thus be readily used for performing an operation with said specific tissue type under optimal conditions.

The present invention is thus also related to a kit of parts for performing an operation on or with one tissue type, comprising:
a) components for preparing a fully defined hydrogel matrix array, so as to create fully defined three-dimensional extracellular matrix conditions that differ from each other in their biological, biophysical and/or biochemical characteristics, said components comprising
   one or more different hydrogel precursor molecules,
   optionally at least one crosslinking agent,
   optionally one or more biologically active molecules,
b) one or more different culture media,

wherein a specific combination of hydrogel features has been preselected for the tissue type to be tested.

The kits according to the present invention are designated for a specific tissue type and can be readily used for performing operations on or with said specific tissue type, such as testing the influence of drugs on said specific tissue type, or the isolation of the grown cells in order to use said grown cells (e.g. 3D cellular structures) in basic scientific research or to implant said cells into a human, for the purposes of personalized or regenerative medicine. The kits according to the present invention are correspondingly indicated, e.g. by instructions provided with said kit, for the specific tissue type with which it is to be used.

Kits of parts are known in the art. Typically they comprise, within a package, one or more containers in which the components defined above are stored separately or together.

According to a preferred embodiment, a hydrogel precursor formulation in the form of an unreacted powder is provided in one container of the kit of parts. Said unreacted powder can be resuspended for use in an appropriate buffer, and dispensed onto a substrate or into discrete volumes of a substrate, preferably a multi-well plate. Said hydrogel precursor formulation in the form of an unreacted powder comprises all the components required for the formation of a hydrogel according to the present invention, i.e. the above discussed one or more different hydrogel precursor molecules, at least one optional crosslinker molecules, and the one or more optional bioactive agent.

The provision of said unreacted powder of said hydrogel precursor formulation is known in the art, e.g. from WO 2011/131642 A1 where lyophilisation was used as means for providing said powder.

### EXEMPLARY EMBODIMENTS

The present invention will now be described below with reference to non-limiting exemplary embodiments and drawings.

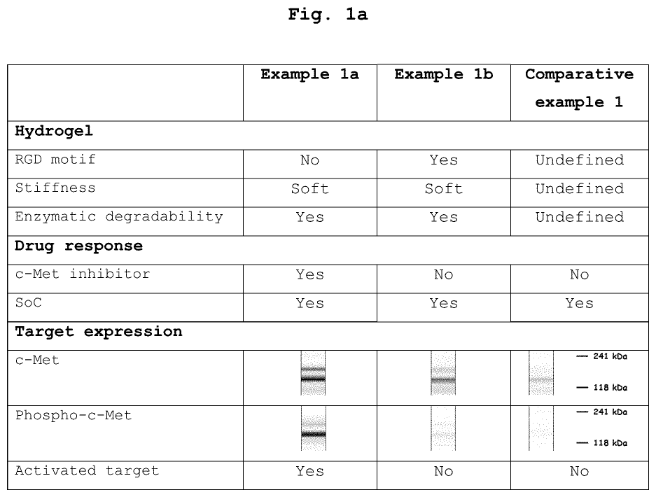

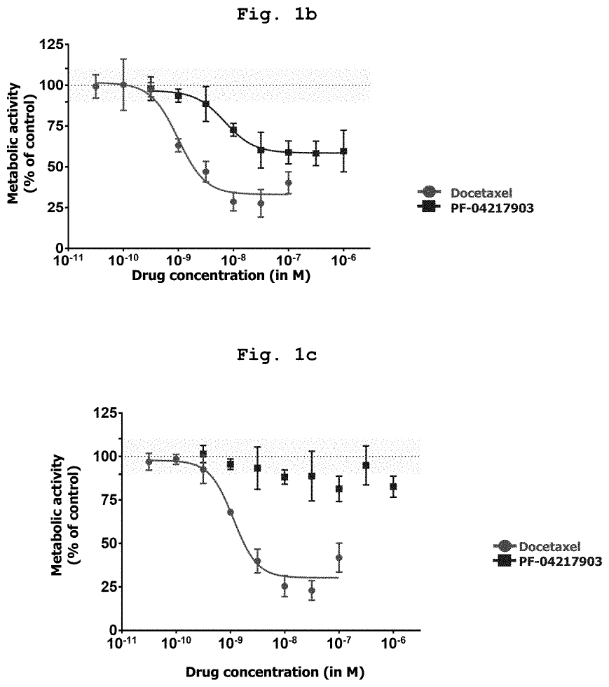

FIG. 1a shows the results of c-met expression in different ex vivo examples and drug testing experiments with non-small cell lung cancer cells overexpressing c-met grown in different gels.

FIG. 1b shows the effect of a SoC treatment and treatment with a c-met inhibitor in an example according to the present invention.

FIG. 1c shows the effect of a SoC treatment and treatment with a c-met inhibitor in a comparative example (Matrigel®).

FIG. 1d shows the results of c-met and EGFR expression in different ex vivo examples.

FIG. 1e shows the effect of a SoC treatment and treatment with EGFR inhibitors in an example according to the present invention.

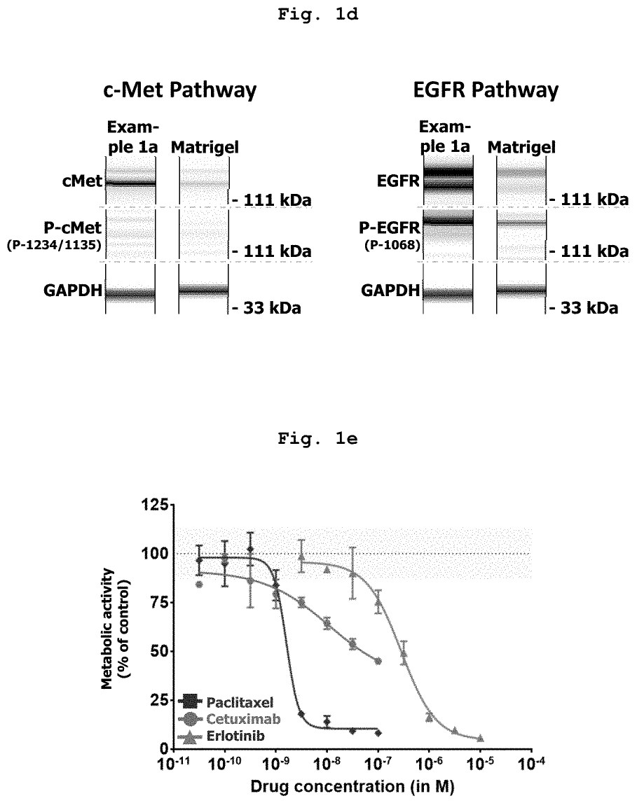

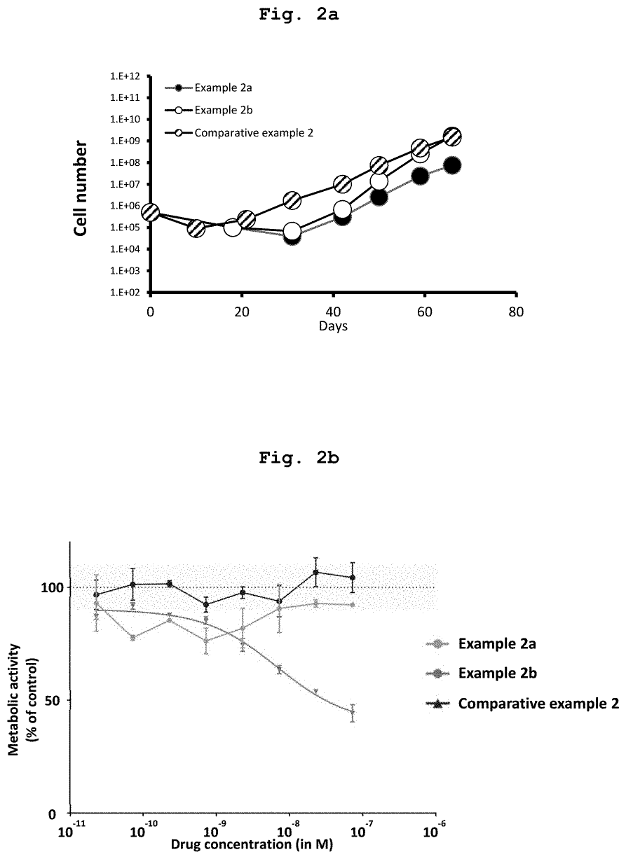

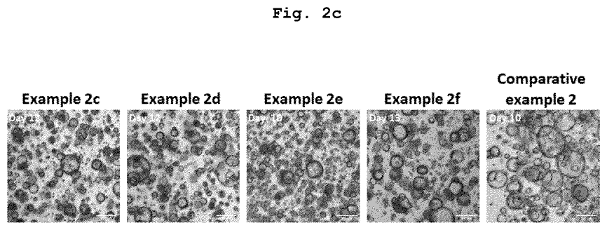

FIG. 2a shows the growth of PDX pancreatic ductal adenocarcinoma (PDAC) cells in different gels.

FIG. 2b shows the drug sensitivity of PDX pancreatic ductal adenocarcinoma (PDAC) cells in different gels.

FIG. 2c shows the growth of PDX pancreatic ductal adenocarcinoma (PDAC) cells in soft and medium gels.

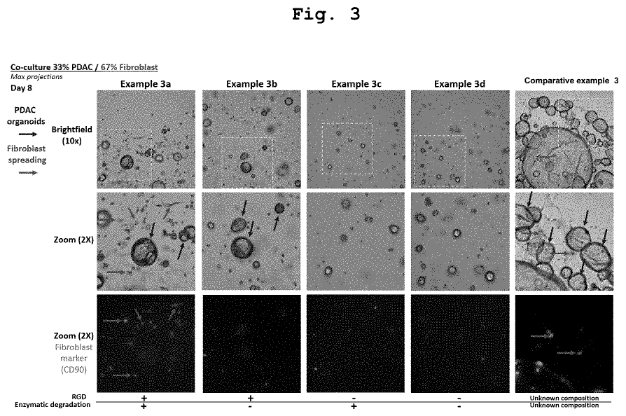

FIG. 3 shows Brightfield images of the results of co-culturing 33% PDAC cells with 67% fibroblasts in different gels.

FIG. 4 shows Brightfield images of the results of human colon cancer organoids grown for 0 and 11 days.

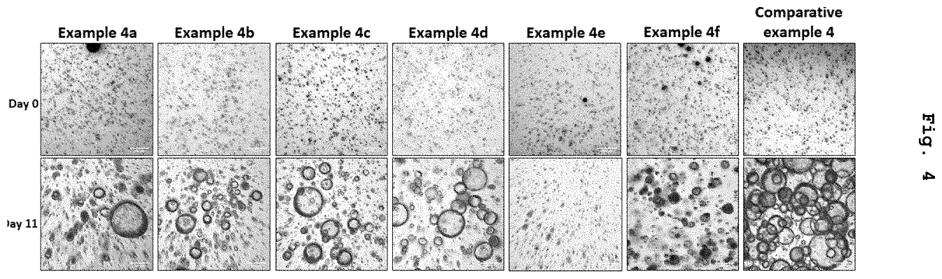

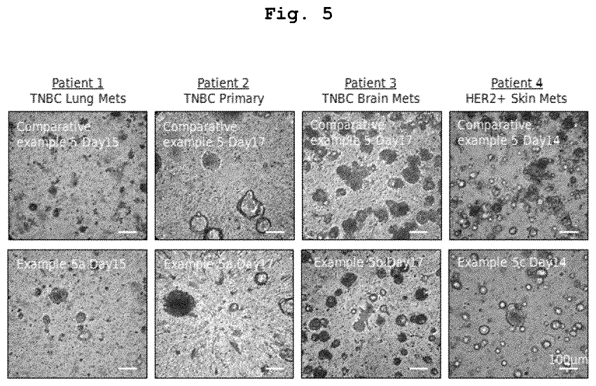

FIG. 5 shows Brightfield images of the results of growth of human primary or metastatic (Mets) breast cancer cells from four patients of either HER2+ or Triple Negative Breast Cancer (TNBC) (from patient-derived xenograft models).

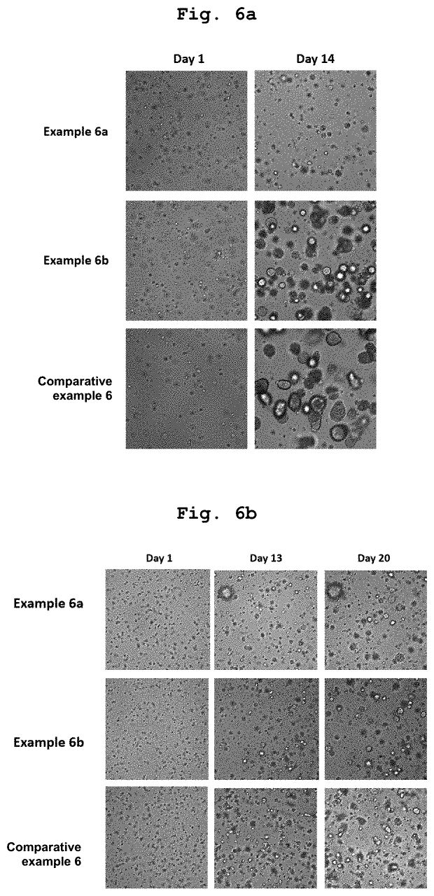

FIG. 6a shows Brightfield images of the results of human healthy prostate cells grown for 1 and 14 days.

FIG. 6b shows Brightfield images of the results of human prostate cancer cells grown for 1, 13 and 20 days.

### LUNG CANCER THERAPY

Well characterized and patient-cell derived preclinical models are essential components to perform reliable translational cancer research, including identifying molecular pathways of oncogenesis and evaluating potential therapeutics.

Tumor cell lines have long existed as a convenient platform for investigation, and numerous cell lines have been well characterized and used for establishing tumors in animal models (xenograft tumors). However, cell line-derived xenograft tumors suffer a lack of predictable relationship between therapeutic responses in preclinical models when compared to responses in human trials and do not accurately recapitulate the tumor microenvironment in a human (Johnson et al., British Journal of Cancer (2001) 84(10), 1424-1431).

Patient-derived tumor xenograft models (PDX) are frequently used for translational cancer research and are assumed to behave consistently over serial passaging. Correlations between histopathological and genotypic characteristics of the original patient samples and PDX models have been well documented (Rubio-Viqueira et al., Clin. Cancer Res. 2006, 12(15), 4652). In addition, PDX models grown over multiple passages maintain a correlation between original human tumor therapeutic responses and the responses in PDX derived from these same patients. However, the throughput of PDX-based screening models is low, and furthermore such screening tests are expensive.

The present invention provides an improved method for cancer research. The present invention provides an improved alternative to PDX models that enables high-throughput screening in a very cost-effective manner.

According to a preferred embodiment, a pre-selection of suitable extracellular matrix conditions for cancer cell growth can be performed by using cells from a PDX and assaying them as described above in the section “preselection”. The histopathological and genotypic characteristics of cells grown ex vivo under such conditions can be correlated with the ones of in vivo established PDX models, and PDX tumor therapeutic responses derived from those PDX models can be used as an in vivo benchmark to evaluate which extracellular matrix conditions can recapitulate in vivo cancer cell behaviour.

As shown in the literature above, the microenvironment (i.e. the extracellular matrix conditions) may influence how cancer cells respond to drug treatments, both in vivo and ex vivo. With the method of the present invention, it is possible to establish ex vivo cell culture conditions for drug screening/testing that are capable to capture the different patient tumor characteristics (e.g. different cancer subtypes), in order to more accurately predict drug treatment outcomes for patients.

This consists in growing cells and testing possible drug treatments on the grown cells ex vivo, using the patient's own cells cultured in a pre-selected range of microenvironments. With the method of the present invention, it is possible to capture the intra- and inter-tumor patient heterogeneity of drug responses (incl. resistance to targeted-therapies).

Currently, the established method in the prior art is still that cells extracted from patient tissues are grown using a single culture condition composed by e.g. the gold-standard Matrigel®. This single condition does not always allow growing patient cells in a way that all features and the possible heterogeneity of the original patient tumors are captured. Also, sometimes some components of the undefined matrix may interfere with the drug response on tested cells.

With the present invention, it is possible to culture patient cells that are then exposed to different drug treatments in order to uncover sensitivities and potential resistance to drug treatments (and underlying mechanisms) that better reflect what is happening in the original patient tumors (e.g. tumor heterogeneity, drug response). With the present invention, it is possible to help selecting or excluding drug treatment for cancer patient and/or to help selecting second line treatments to overcome the resistance to previous treatment(s).

Following this approach, it could be shown that preselected conditions that are suitable for testing the effect of c-Met inhibitors on non-small cell lung cancer (NSCLC) cells overexpressing c-Met are characterized by the absence of any RGD adhesion motif in the hydrogel (see example 1 below).

This is particularly surprising since from the prior art the opposite result (necessity of presence of RGD adhesion motif in the extracellular matrix conditions) would have been expected. In Mitra et al. (Oncogene 2011, March 31; 30(13): 1566-1576) it was shown that c-Met can be activated independently of its ligand (HGF) via the fibronectin-mediated activation of α5β1-integrin leading to its interaction with c-Met receptor. Inhibition of α5β1-integrin decreased the phosphorylation of c-Met, both in vitro and in vivo (ovarian cancer lines). The crosstalk between integrin β1 with c-Met was also explored for NSCLC in Ju et al. (Cancer Cell International 2013, 13:15). This article showed that interaction of integrin β1 with c-Met induces c-Met activation (i.e. phosphorylation) and permits cancer cells sensitive to inhibition of EGFR receptor to become resistant to EGFR targeted drugs by bypassing the EGF pathway. Both articles show that c-Met can interact with integrin β1, which is a known RGD linker. They also demonstrate that this interaction resulted in c-Met phosphorylation and activation of its downstream pathway (FAK, AKT), inducing cell proliferation and increased survival. In summary, both articles clearly highlight the relationship between c-Met receptor and fibronectin.

According to the present invention, however, it could be shown that the presence of a RGD motif in the hydrogel matrix led to a downregulation of the c-Met receptor and the absence of activated c-Met receptor (i.e. phosphorylated receptor) in NSCLC cells, resulting in their resistance against treatment with a c-Met inhibitor. It was an important finding of the present invention that the absence of any RGD adhesion motif in the hydrogel provided the correct preselected conditions for identifying suitable drug candidates for NSCLC cancer cells exhibiting an activated c-met receptor. On the other hand, NSCLC cancer cells growing in the presence of a RGD motif do not possess and do not rely on an activated c-met receptor, and this indicates that they may also have to be treated with other drug candidates than a c-met inhibitor (possibly along with a c-met inhibitor). Without the use of the “preselected growth conditions”, it would not have been possible to understand that these cells may rely on other mechanisms of growth than c-Met. One of the major added value of using said “preselected growth conditions” compared to single growth conditions as used in the prior art, is that it enables uncovering the heterogeneity (e.g. genetic, phenotypic) of the specific cancer tissue and cancer type, as well as a better possible range of treatments that are needed to cure said cancer. This has relevance in personalize medicine as well as drug development applications.

Thus, according to this embodiment, the present invention is related to a method of testing the influence of c-Met inhibitors on lung cancer cells, preferably non-small cell lung cancer cells, overexpressing c-Met, comprising the steps of:
a) providing preselected extracellular matrix conditions comprising a fully defined non self-degradable hydrogel matrix array with discrete volumes prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said lung cancer cells, preferably non-small cell lung cancer cells, so as to create fully defined three-dimensional extracellular matrix conditions that differ from each other in their biological, biophysical and/or biochemical characteristics;
b) allowing said lung cancer cells, preferably non-small cell lung cancer cells, to grow in said discrete volumes of said hydrogel matrix in the presence of one or more different culture media, preferably comprising FBS (serum) or Wnt agonist such as R-spondin, especially preferred also comprising FGF-7, FGF-10, HGF, and a TGF-β inhibitor;
c) adding a drug targeting c-Met receptor or c-Met pathway to the cells grown in said discrete volumes of said hydrogel matrix;

wherein said crosslinking agent and said optional bioactive agent do not comprise any RGD motif.

According to a very preferred embodiment, the present invention is related to a method of testing the influence of c-Met inhibitors and other drugs on lung cancer cells, preferably non-small cell lung cancer cells, comprising the steps of:
a) providing, in a first array of a substrate, preselected extracellular matrix conditions comprising a fully defined non self-degradable hydrogel matrix array with discrete volumes prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said lung cancer cells, preferably non-small cell lung cancer cells, so as to create fully defined three-dimensional extracellular matrix conditions that differ from each other in their biological, biophysical and/or biochemical characteristics, wherein said crosslinking agent and said optional bioactive agent do not comprise any RGD motif, and providing, in a second array of the substrate, preselected extracellular matrix conditions that differ from the preselected extracellular matrix conditions in the first array by the presence of an RGD motif in said crosslinking agent and/or said optional bioactive agent;
b) allowing said lung cancer cells, preferably non-small cell lung cancer cells, to grow in said discrete volumes of said hydrogel matrix in the first array and second array in the presence of one or more different culture media, preferably comprising FBS (serum) or Wnt agonist such as R-spondin, especially preferred also comprising FGF-7, FGF-10, and a TGF-β inhibitor;
c) adding a drug targeting c-Met receptor or c-Met pathway to the cells grown in said discrete volumes of said hydrogel matrix in the first array and second array;
d) adding at least one other drug, preferably a EGFR-receptor inhibitor, to the cells grown in said discrete volumes of said hydrogel matrix in the first array and second array, into wells where no drug targeting c-Met receptor or c-Met pathway has been added.

Preferably, said hydrogel matrix array has a soft or medium stiffness in the range of 50-2000 Pa.

Preferably, said PEG hydrogel precursor molecule is PEG-VS (Polyethylene glycol with terminal vinylsulfone moieties), especially preferable 4-arm or 8-arm PEG-VS.

More preferably, said fully defined non self-degradable hydrogel matrix array is prepared by crosslinking PEG-VS, especially preferable 4-arm or 8-arm PEG-VS, with a peptide comprising at least two, preferably two cysteine moieties as a crosslinking agent, wherein said crosslinking agent does not comprise any RGD motif. Especially preferred, no bioactive ligand is attached to the hydrogel matrix.

As an optional bioactive ligand, a ligand comprising a bioactive motif except any RGD adhesion motif may be used.

Preferably, said optional bioactive ligand is selected from the group consisting of natural laminins, for example laminin-111, in particular mouse laminin-111, recombinant laminin isoforms, and biofunctional fragments thereof. Examples of suitable recombinant laminin isoforms are laminin-111, laminin-211, laminin-332, laminin-411, laminin-511, or laminin-521.

As an optional bioactive ligand, a ligand comprising glycosaminoglycans such as hyaluronic acid and hyaluronan may be used. Examples of hyaluronic acid are hyaluronic acid 50k, hyaluronic acid 1000k, hyaluronate thiol 50k or hyaluronate thiol 1000k.

Preferably, said culture medium is characterized by the presence of FBS (serum) or Wnt agonists such as R-spondin. According to a preferred embodiment, a culture medium may be used that is adapted from the medium described in Sachs et al. (The EMBO Journal e 100300|2019). The preferred culture medium comprises AdDMEM/F12 medium supplemented with glutamine, Noggin, EGF, fibroblast growth factor 7 and 10 [FGF7 and FGF10], HGF, R-spondin-conditioned medium, Primocin, penicillin/streptomycin, N-acetyl-L-cysteine, Nicotinamide, A83-01, SB202190 (p38-inhibitor), Y-27632 (rock inhibitor), B27 supplement and HEPES. Other media like the ones described in Lancaster et al. (Nat Bi otechnol 2017 35(7): 659-666), or the commercially available culture media from PromoCell (Small Airway Epithelial Cell Growth Medium (C-39175)) or from Invitrogen (StemPro™ hESC SFM) may be used.

Especially preferable, said lung cancer cells, preferably nonsmall cell lung cancer cells, overexpressing c-Met are from freshly isolated or frozen human cells from a biopsy or tissue resection of a human or from patient-derived xenograft (PDX) tissue.

With said method, it is possible to grow, expand and subsequently test said lung cancer cells, preferably non-small cell lung cancer cells, in a selected medium under extracellular matrix conditions that recapitulate drug results observed in vivo.

According to an especially preferred embodiment, the extracellular matrix conditions are chosen such that the use of a naturally-derived matrix such as Matrigel® can be completely avoided.

The present invention provides a method with preselected extracellular matrix conditions that sustain the growth as well as the expansion of lung cancer cells, preferably NSCLC cells, using a fully defined or preferably fully synthetic hydrogel matrix. The method allows the reproduction of target expression and drug responses observed in vivo in PDX lung models and not achieved with Matrigel®. Preselection is important, since different ex vivo conditions can promote different drug responses confirming that using a single culture condition may not reflect what is happening in the original patient tumor.

According to this embodiment, the present invention is also related to a kit of parts for testing the influence of c-Met inhibitors on lung cancer cells, preferably non-small cell lung cancer cells, overexpressing c-Met, comprising:
a) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and said bioactive agent do not comprise any RGD motif;
b) one or more different culture media, preferably comprising FBS (serum) or Wnt agonist such as R-spondin.

According to this embodiment, the present invention is also related to a kit of parts for testing the influence of c-Met inhibitors on lung cancer cells, preferably non-small cell lung cancer cells, overexpressing c-Met, comprising:
a) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and said bioactive agent do not comprise any RGD motif;
b) one or more different culture media, preferably comprising FBS (serum) or Wnt agonist such as R-spondin
c) optionally, cells from a cell repository/biobank that have been created using the same extracellular matrix conditions.

According to another preferred variant of this embodiment, the present invention is also related to a kit of parts for testing the influence of c-Met inhibitors and other drugs on lung cancer cells, preferably non-small cell lung cancer cells, comprising:
a) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and said bioactive agent do not comprise any RGD motif;
b) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and/or said bioactive agent comprise an RGD motif;
c) one or more different culture media, preferably comprising FBS (serum) or Wnt agonist such as R-spondin.

According to another preferred variant of this embodiment, the present invention is also related to a kit of parts for testing the influence of c-Met inhibitors and other drugs on lung cancer cells, preferably non-small cell lung cancer cells, comprising:
a) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and said bioactive agent do not comprise any RGD motif;
b) components for preparing a fully defined non self-degradable hydrogel matrix array so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein said crosslinking agent and/or said bioactive agent comprise an RGD motif;
c) one or more different culture media, preferably comprising FBS (serum) or Wnt agonist such as R-spondin;
d) optionally, cells from a cell repository/biobank that have been created using the same extracellular matrix conditions.

With the method and kit of the present invention, it is also possible to culture and test drugs on additional lung cancer cells that bear different disease features (e.g. different lung cancer subtypes, mutations such as EGFR, KRAS or PI3 kinase mutations). By the same manner as described above, extracellular matrix conditions can be preselected for those other cells.

According to a particularly preferred embodiment, the hydrogel does not comprise any RGD binding site, and especially preferred no integrin binding site at all.

As can be seen from example 1 and related FIG. 1 discussed below, when testing drugs on their activity against non-small cell lung cancer cells overexpressing c-Met, preselection of the correct conditions is of paramount importance. With Matrigel®, i.e. the standard matrix in the prior art, it is not possible to identify a drug candidate targeting c-Met. As has been found in comparative example 1, this is probably because under conditions of employing Matrigel® the drug target c-Met is not sufficiently expressed and is not activated. Accordingly, under conditions of employing Matrigel® it is not possible to identify suitable drug candidates that act against the most important target in those lung cancer cells, i.e. the overexpressed c-Met.

Also, in example 1 and related FIG. 1 discussed below it was shown that targeting c-Met is actually a key feature for inhibiting growth of non-small cell lung cancer cells overexpressing c-Met.

In addition, with the preselected conditions of this embodiment of the present invention, it is also possible to better identify an optimal treatment regime for a specific patient. It was found that certain patients did not respond to drug treatment with a c-met inhibitor alone, presumably because such patients had a cell phenotype that could compensate for c-met inhibition. With the preselected conditions of this embodiment of the present invention, it is possible to screen for drug combination treatment using a c-met inhibitor together with another drug type. As discussed above, this is not possible with prior art conditions using Matrigel®. The present invention provides a better predictability for patient response.

The preselected extracellular matrix conditions of this embodiment can also be used in a method for testing the efficiency of therapies for a cancer patient, comprising the steps:
a) providing freshly isolated or frozen lung cancer cells, preferably non-small cell lung cancer cells, from a biopsy or a tissue resection of a cancer patient;
b) establishing and expanding organoids from said cells, and applying one or more drugs to said organoids by the method described above;
c) comparing the activity of the one or more drugs applied in step b) with the result of the treatment of said patient with one of said drugs applied in step b);
d) and/or providing drug activity results on patient organoids and corresponding genetic and phenotypic data of the disease to support physician in making decisions on how to treat the patient.

Patient biopsies or resections dedicated for the isolation of lung cancer cells, preferably non-small cell lung cancer (NSCLC) cells, to establish organoids in step b), can be collected during a standard diagnostic procedure and subsequently transported to the site where step b) is to be conducted.

Step b) of this method is conducted as described above, i.e. by providing preselected extracellular matrix conditions comprising a fully defined non self-degradable hydrogel matrix array with discrete volumes having a stiffness in the range of 50 to 2000 Pa and prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said lung cancer cells, preferably non-small cell lung cancer cells, so as to create fully defined three-dimensional extracellular matrix conditions that differ from each other in their biological, biophysical and/or biochemical characteristics;

allowing said lung cancer cells, preferably non-small cell lung cancer cells, to grow in said discrete volumes of said hydrogel matrix in the presence of one or more different culture media preferably comprising FBS (serum) or Wnt agonist such as R-spondin, especially preferred also comprising FGF-7, FGF-10, and a TGF-β inhibitor;

and adding one or more drugs to the cells grown in said discrete volumes of said hydrogel matrix;

wherein said crosslinking agent and said optional bioactive agent do not comprise a RGD motif.

The one or more drugs added to the cells grown in said discrete volumes of said hydrogel matrix comprise the drug(s) used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests.

Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Patient-Derived Organoids (PDO) in Precision Medicine

Cancer is a multifactorial disease that results from both genetic and epigenetic transformation of normal cells, leading to abnormal proliferation. Conventional cancer treatments include surgical resection, radiotherapy, non-specific or targeted chemotherapies and immunotherapy to inhibit cell division or induce apoptosis of cancer cells.

Different cancer types respond to treatment in different ways, and therefore some cancer types can be treated better than others. Despite the development of potent chemotherapeutics and oncogene-specific targeted drugs, durable or long-lasting cure of this disease has not been achieved for many patients.

Recent improvements of DNA sequencing technologies allow the fast identification of specific genome mutations of patient tumors with the potential of tailoring cancer therapies based on molecular profiles of tumors. Significant improvements were shown in the treatment of leukemia, lung and melanoma cancers (Druker et al. (N Engl J Med, Vol. 344, No. 14 (2001), 1031), Lynch et al. (N Engl J Med 350; 21 (2004), 2129), Flaherty et al. (N Engl J Med 2010; 363:809-19). However, the clinical benefit of genome-guided precision medicine is still highly debatable (Le Tourneau et al. (www.thelancet.com/oncology, Published online Sep. 3, 2015, http://dx.doi.org/10.1016/S1470-2045(15)00188-6), Prasad (Nature 537 (2016), S63), Letai (NATURE MEDICINE, VOLUME 23|NUMBER 9|September 2017, 1028). Recent clinical trials assessing the rate of assigning patients with solid tumors to targeted therapies showed that only part of them (10-50%) bear mutations matching available clinically validated and approved therapies (Letai 2017, Sicklick (Nature Medicine https://doi.org/10.1038/s41591-019-0407-5 (2019)). Besides this, two fundamental biological aspects impair the efficiency of genome-guided precision medicine:
resistance to the specific therapy due to the intra-patient genetic and phenotypic heterogeneity of cancer cells (presence of divergent subclones) (Tannock et al. (N Engl J Med 375; 13 (2016), 1289), Flavahan et al., (Science 357, 266 (2017));
insufficient biological understanding of the tumor microenvironment effect in modulating the drug response (Friedmann et al. (Nature Reviews Cancer AOP, published online 5 Nov. 2015; doi:10.1038/nrc4015 2015)).

Screens of drugs on patient-derived cells (functional precision medicine) could address these limitations and be complementary to genomics and pathological data to support the prediction of patient outcome and therefore guiding the decision-making therapy process.

Novel in vitro tumor biology models that recapitulate the in vivo tumor microenvironment, such as Patient Derived Organoids (PDO), have the advantage of growing in a 3D environment, reproducing the spatial architecture of the original tissue. Organoids are miniature 3D in vitro structures grown from patient-derived cells that mimic key features and functions of its original healthy or diseased tissue. A variety of PDO have been established for many tumors including—but not limited to—colorectal cancer (Sato et al. (Nature vol. 469 (2011), 415), van de Wetering et al. (Cell 161 (2015), 933-945), pancreas ductal adenocarcinoma (Boj et al. (Cell 160, 324-338, Jan. 15, 2015), Huang et al. (Nature medicine, published online 26 Oct. 2015; doi:10.1038/nm.3973), breast cancer (Sachs et al. (Cell 172 2018, 1-14) and lung cancer (Sachs et al. (The EMBO Journal e 100300|2019). Overall, these studies showed that PDO can maintain the same genetic driver mutations identified in the primary tumor.

Recently, patient organoids derived from different locations of the same tumor were used to study the nature and extent of intra-tumor heterogeneity and to assess their response to a panel of drugs (Roerink et al. Nature 556, 457-462, 2018). Significant differences in responses to drugs between closely related cells of the same tumor were observed.

Prospective use of organoids as functional diagnostic tool in clinic has been shown already for rectal cancer (Ganesh et al., Nature Medicine, 10, 1067-1614(2019)) metastatic colorectal cancer (Vlachogiannis et al., Science 359, 920-926 (2018) and Ooft et al., Science Translational Medicine, 11, (2019), DOI: 10.1126/scitranslmed.aay2574), pancreatic cancer (Tiriac, CANCER DISCOVERY, SEPTEMBER 2018, DOI: 10.1158/2159-8290.CD-18-0349) and appendiceal cancer (Votanopoulos et al., Ann Surg Oncol (2019) 26:139-147). In these studies, the drug response of the PDOs correlates with the outcome of the same treatment on patients from which the organoids were derived.

Despite these promising results of the PDO drug responses matching the corresponding patient outcomes, these studies are confined to a limited number of patients, and the methods used rely on a basal membrane extract (BME) with undefined composition and batch to batch variability, such as Matrigel®. This represents a significant limitation in the standardization of the PDO for translation to clinically relevant applications. Also, only single culture conditions were employed for each type of cancer, regardless of possible differences in genetic and/or phenotypic tumor features, which also include, but is not limited to the biomarker expression, that may require different extracellular matrix conditions. This may favour the growth of specific cell populations or inducing the expression of only some phenotypes during ex vivo cell expansion (WO 2010/090513 A2; WO 2016/015158 A1; WO 2015/173425 A1) and therefore failing to mimic in vivo tumor characteristics and drug responses. This has been outlined above with respect to lung cancer cells, preferably non-small cell lung cancer cells, overexpressing c-Met.

In order to overcome the limitations of naturally-derived matrices such as Matrigel®, fully-defined and also synthetic hydrogels have been already investigated to grow a variety of tissues, including intestinal and colon organoids from mouse and human origins (Gjorevski et al., Designer matrices for intestinal stem cell and organoid culture, Nature, Vol 539, 24 Nov. 2016, 560-56, WO 2017/037295 A1; or Cruz-Acuna et al., Synthetic hydrogels for human intestinal organoid generation and colonic wound repair, Nature cell biology, advanced online publication published online 23 Oct. 2017; DOI: 10.1038/ncb3632, 1-23, WO 2018/165565 A1) and more recently from appendiceal, pancreatic and mesothelioma cancer patient cells (Votanopoulos 2019 (above), Broguiere et al., Adv. Mater. 2018, U.S. Pat. No. 1,801,621 (2018), Mazzocchi et al., SCIENTIFIC Reports (2018) 8:2886 DOI:10.1038/s41598-018-21200-8).

Although some of these studies are using a fully-defined or fully synthetic matrix, they still rely on the use of single culture condition regardless of the tumor feature.

### Pancreatic Cancer

With the present invention, it is possible to culture patient pancreatic cells, preferably pancreatic ductal adenocarcinoma (PDAC) cells, under conditions that sustain the growth and expansion of these cells. Subsequently, the cells are exposed to different drug treatments in order to select an efficient drug treatment for the cancer patient.

According to the present invention, it could be shown that PDAC cells could be well cultured and tested using a combination of a fully defined soft (50-1000 Pa stiffness) or medium (1000-2000 Pa) or hard (2000-3000 Pa) non self-degradable hydrogel matrix comprising at least one RGD adhesion motif and a culture medium, preferably comprising Wnt agonists such as R-spondin and Wnt 3a.

Thus, according to this embodiment, the present invention is related to a method of testing the influence of drugs on pancreatic ductal adenocarcinoma (PDAC) cells, comprising the steps of:
a) providing preselected extracellular matrix conditions comprising a fully defined non self-degradable hydrogel matrix array with discrete volumes having a stiffness in the range of 50 to 3000 Pa, preferably 50 to 2000 Pa and most preferably 50 to 1000 Pa, and prepared by crosslinking, onto a substrate or into discrete volumes of a substrate, preferably a multiwell plate, different combinations of one or more different PEG hydrogel precursor molecules, in the presence of optionally one or more biologically active molecules, at least one crosslinking agent, and said pancreatic ductal adenocarcinoma cells, so as to create fully defined three-dimensional extracellular matrix conditions that differ from each other in their biological, biophysical and/or biochemical characteristics;
b) allowing said pancreatic ductal adenocarcinoma cells to grow in said discrete volumes of said hydrogel matrix in the presence of one or more different culture media, preferably comprising Wnt agonists such as R-spondin and Wnt 3a;
c) adding one or more drugs to the cells grown in said discrete volumes of said hydrogel matrix;

wherein at least one of said crosslinking agent and/or said optional bioactive agent comprises a RGD motif.

Preferably, said PEG hydrogel precursor molecule is PEG-VS (Polyethylene glycol with terminal vinylsulfone moieties), especially preferable 4-arm or 8-arm PEG-VS.

More preferably, said fully defined non self-degradable hydrogel matrix array is prepared by crosslinking PEG-VS, especially preferable 4-arm or 8-arm PEG-VS, with a peptide comprising at least two, preferably two cysteine moieties as a crosslinking agent, wherein said crosslinking agent may comprise a RGD motif.

As an optional bioactive ligand, a ligand comprising a bioactive motif including a RGD adhesion motif may be used. Examples of suitable RGD motifs are RGD, RGDS, RGDSP, RGDSPG, RGDSPK, RGDTP, RGDSPASSKP, PHSRNSGSGSGSGSGRGDSPG or any cyclic RGD motifs such as cyclo(RGDfC), but principally any known and successfully employed RGD sequences, in the field of hydrogels and cell culture, could be used.

Preferably, said optional bioactive ligand is selected from the group consisting of natural laminins, for example laminin-111, in particular mouse laminin-111, recombinant laminin isoforms, and biofunctional fragments thereof. Examples of suitable recombinant laminin isoforms are laminin-111, laminin-211, laminin-332, laminin-411, laminin-511, or laminin-521, laminin-511 being preferred.

As an optional bioactive ligand, a ligand comprising a bioactive motif including a collagen peptide motif may be used. Example of a suitable collagen peptide is DGEA.

As an optional bioactive ligand, a ligand comprising glycosaminoglycans such as hyaluronic acid and hyaluronan may be used. Examples of hyaluronic acid are hyaluronic acid 50k, hyaluronic acid 1000k, hyaluronate thiol 50k or hyaluronate thiol 1000k.

Preferably, said culture medium is characterized by the presence of Wnt agonists such as R-spondin and Wnt 3a. According to a preferred embodiment, a culture medium may be used that is described in Boj et al. (Cell 160, 324-338, Jan. 15, 2015), p. 335, right col., 2^{nd} para, or Huang et al. (Nature medicine, published online 26 Oct. 2015; doi:10.1038/nm.3973). Especially preferred is the culture medium adapted from Boj et al., which comprises AdDMEM/F12 medium supplemented with HEPES, Glutamax, penicillin/streptomycin, B27, Primocin, N-acetyl-L-cysteine, Wnt3a-conditioned medium [50% v/v] or recombinant protein [100 ng/ml], RSPO1-conditioned medium [10% v/v] or recombinant protein [500 ng/ml], Noggin-conditioned medium [10% v/v] or recombinant protein [0.1 μg/ml], epidermal growth factor [EGF, 50 ng/ml], Gastrin [10 nM], fibroblast growth factor 10 [FGF10, 100 ng/ml], Nicotinamide [10 mM], Prostaglandin E2 [PGE2, 1 μM] and A83-01 [0.5 μM].

Especially preferable, said pancreatic ductal adenocarcinoma cells are from freshly isolated or frozen cells from a biopsy or tissue resection of a human or from patient-derived xenograft (PDX) tissue.

With said method, it is possible to grow, expand and subsequently test said pancreatic ductal adenocarcinoma cells in a selected medium under extracellular matrix conditions that recapitulate drug results observed in vivo.

According to an especially preferred embodiment, the extracellular matrix conditions are chosen such that the use of a naturally-derived matrix such as Matrigel® can be completely avoided.

According to this embodiment, the present invention is also related to a kit of parts for testing the influence of drugs on pancreatic ductal adenocarcinoma cells, comprising:
a) components for preparing a fully defined non self-degradable hydrogel matrix array having a stiffness in the range of 50 to 3000 Pa, preferably 50 to 2000 Pa and most preferably 50 to 1000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein at least one of said crosslinking agent and/or said optional bioactive agent comprises a RGD motif;
b) one or more different culture media, preferably comprising Wnt agonists such as R-spondin and Wnt 3a.

According to this embodiment, the present invention is also related to a kit of parts for testing the influence of drugs on pancreatic ductal adenocarcinoma cells, comprising:
a) components for preparing a fully defined non self-degradable hydrogel matrix array having a stiffness in the range of 50 to 3000 Pa, preferably 50 to 2000 Pa and most preferably 50 to 1000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein at least one of said crosslinking agent and/or said optional bioactive agent comprises a RGD motif;
b) one or more different culture media, preferably comprising Wnt agonists such as R-spondin and Wnt 3a;
c) optionally, cells from a cell repository/biobank that have been created using the same extracellular matrix conditions.

The preselected extracellular matrix conditions of this embodiment can also be used in a method for testing the efficiency of therapies for a cancer patient, comprising the steps:
a) providing freshly isolated or frozen pancreatic ductal adenocarcinoma cells from a biopsy or a tissue resection of a cancer patient;
b) establishing and expanding organoids from said cells, and applying one or more drugs to said organoids by the method described above;
c) comparing the activity of the one or more drugs applied in step b) with the result of the treatment of said patient with one of said drugs applied in step b);
d) and/or providing drug activity results on patient organoids and corresponding genetic and phenotypic data of the disease to support physician in making decisions on how to treat the patient.

Patient biopsies or resections dedicated for the isolation of pancreatic ductal adenocarcinoma (PDAC) cells to establish organoids in step b), can be collected during a standard diagnostic procedure and subsequently transported to the site where step b) is to be conducted.

Step b) of this method is conducted as described above, i.e. by providing preselected extracellular matrix conditions comprising a fully defined non self-degradable hydrogel matrix array with discrete volumes having a stiffness in the range of 50 to 3000 Pa, preferably 50 to 2000 Pa and most preferably 50 to 1000 Pa and prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said pancreatic ductal adenocarcinoma cells, so as to create fully defined three-dimensional extracellular matrix conditions that differ from each other in their biological, biophysical and/or biochemical characteristics;

allowing said pancreatic ductal adenocarcinoma cells to grow in said discrete volumes of said hydrogel matrix in the presence of one or more different culture media preferably comprising Wnt agonists such as R-spondin and Wnt 3a;

and adding one or more drugs to the cells grown in said discrete volumes of said hydrogel matrix;

wherein at least one of said crosslinking agent and/or said optional bioactive agent comprises a RGD motif.

The one or more drugs added to the cells grown in said discrete volumes of said hydrogel matrix comprise the drug(s) used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests.

Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Co-Culturing of PDAC Cells

According to another preferred embodiment of the present invention, cancer cells and preferably pancreatic ductal adenocarcinoma (PDAC) cells can be co-cultured in combination with stromal cells, preferably fibroblasts. For this embodiment, the hydrogel matrix is preselected as being a preferably non self-degradable PEG hydrogel having a stiffness in the range of 50 to 3000 Pa, preferably 50 to 2000 Pa and most preferably 50 to 1000 Pa, wherein at least one of the crosslinking agents comprises an enzymatically degradable motif, preferably a MMP-sensitive motif, and wherein at least one of the crosslinking agent and/or said optional bioactive agent comprise a RGD motif.

Preferably, the culture medium to be used in said embodiment comprises Wnt agonists such as R-spondin and Wnt 3a and preferably additionally FBS.

Thus, according to this embodiment, the present invention is related to a method of testing cancer cells, preferably pancreatic ductal adenocarcinoma (PDAC) cells, that have been co-cultured with stromal cells, preferably fibroblasts, comprising the steps of:
a) providing preselected extracellular matrix conditions comprising a fully defined, preferably non self-degradable hydrogel matrix array with discrete volumes having a stiffness in the range of 50 to 3000 Pa, preferably 50 to 2000 Pa and most preferably 50 to 1000 Pa and prepared by crosslinking, onto a substrate or into discrete volumes of a substrate, preferably a multi-well plate, different combinations of one or more different PEG hydrogel precursor molecules, in the presence of optionally one or more biologically active molecules, at least one crosslinking agent, and said cancer cells, preferably pancreatic ductal adenocarcinoma (PDAC) cells, and said stromal cells, preferably fibroblasts, so as to create fully defined three-dimensional extracellular matrix conditions that differ from each other in their biological, biophysical and/or biochemical characteristics;
b) allowing said pancreatic ductal adenocarcinoma cells and stromal cells, preferably fibroblasts, to grow in said discrete volumes of said hydrogel matrix array in the presence of one or more different culture media, preferably comprising Wnt agonists such as R-spondin and Wnt 3a, and preferably additionally FBS;
c) adding one or more drugs to the cells grown in said discrete volumes of said hydrogel matrix;

wherein the at least one crosslinking agent comprises an enzymatically degradable motif, preferably a MMP-sensitive motif, and wherein at least one of the crosslinking agent and/or said optional bioactive agent comprise a RGD motif.

According to a preferred embodiment, said method is carried out such that at least two different arrays are provided, wherein the arrays differ with respect to the presence or absence of an enzymatically degradable motif, preferably a MMP-sensitive motif. In the array where said enzymatically degradable motif, preferably MMP-sensitive motif, is present, the PDAC cells can be co-cultured with the stromal cells, preferably fibroblasts. In the array where said enzymatically degradable motif, preferably MMP-sensitive motif, is not present, the PDAC cells are grown in a single culture that impairs the growth of stromal cells such as fibroblasts.

Preferably, said PEG hydrogel precursor molecule is PEG-VS (Polyethylene glycol with terminal vinylsulfone moieties), especially preferable 4-arm or 8-arm PEG-VS. According to another preferred embodiment, a self-degradable PEG may be prepared from one or more PEG-Acr precursor molecules and used alone or in combination with a PEG-VS precursor molecule.

More preferably, said fully defined, preferably non self-degradable hydrogel matrix array is prepared by crosslinking PEG-VS, especially preferable 4-arm or 8-arm PEG-VS, with a peptide comprising at least two, preferably two cysteine moieties as a crosslinking agent, wherein said crosslinking agent comprises an enzymatically degradable motif, preferably a MMP-sensitive motif, and additionally may comprise a RGD motif.

As an optional bioactive ligand, a ligand comprising a bioactive motif including a RGD adhesion motif may be used. Examples of suitable RGD motifs are RGD, RGDS, RGDSP, RGDSPG, RGDSPK, RGDTP, RGDSPASSKP, PHSRNSGSGSGSGSGRGDSPG or any cyclic RGD motifs such as cyclo(RGDfC), but principally any known and successfully employed RGD sequences, in the field of hydrogels and cell culture, could be used.

Preferably, said optional bioactive ligand is selected from the group consisting of natural laminins, for example laminin-111, in particular mouse laminin-111, recombinant laminin isoforms, and biofunctional fragments thereof. Examples of suitable recombinant laminin isoforms are laminin-111, laminin-211, laminin-332, laminin-411, laminin-511, or laminin-521, laminin-511 being preferred.

As an optional bioactive ligand, a ligand comprising a bioactive motif including a collagen peptide motif may be used. Example of a suitable collagen peptide is DGEA.

As an optional bioactive ligand, a ligand comprising glycosaminoglycans such as hyaluronic acid and hyaluronan may be used. Examples of hyaluronic acid are hyaluronic acid 50k, hyaluronic acid 1000k, hyaluronate thiol 50k or hyaluronate thiol 1000k.

Preferably, said culture medium is characterized by the presence of Wnt agonists such as R-spondin and Wnt 3a, and preferably additionally FBS. According to a preferred embodiment, a culture medium may be used that is described in Boj et al. (Cell 160, 324-338, Jan. 15, 2015), p. 335, right col., 2^{nd} para, or Huang et al. (Nature medicine, published online 26 Oct. 2015; doi:10.1038/nm.3973). Especially preferred is the culture medium adapted from Boj et al., which comprises AdDMEM/F12 medium supplemented with HEPES, Glutamax, penicillin/streptomycin, B27, Primocin, N-acetyl-L-cysteine, Wnt3a-conditioned medium [50% v/v] or recombinant protein [100 ng/ml], RSPO1-conditioned medium [10% v/v] or recombinant protein [500 ng/ml], Noggin-conditioned medium [10% v/v] or recombinant protein [0.1 μg/ml], epidermal growth factor [EGF, 50 ng/ml], Gastrin [10 nM], fibroblast growth factor 10 [FGF10, 100 ng/ml], Nicotinamide [10 mM], Prostaglandin E2 [PGE2, 1 μM] and A83-01 [0.5 μM].

Especially preferable, said pancreatic ductal adenocarcinoma cells are from freshly isolated or frozen cells from a biopsy or tissue resection of a human or from patient-derived xenograft (PDX) tissue, or from patient-derived organoids (PDO), optionally pre-established in BME, such as Matrigel®.

Preferably, said stromal cells are isolated from patient. Especially preferable, said stromal cells are fibroblasts.

With said method, it is possible to grow, expand and subsequently test said pancreatic ductal adenocarcinoma cells in co-culture with stromal cells in a selected medium under extracellular matrix conditions that recapitulate drug results observed in vivo.

According to an especially preferred embodiment, the extracellular matrix conditions are chosen such that the use of a naturally-derived matrix such as Matrigel® can be completely avoided.

According to this embodiment, the present invention is also related to a kit of parts for testing the influence of drugs on cancer cells, preferably pancreatic ductal adenocarcinoma (PDAC) cells, that have been co-cultured with stromal cells, preferably fibroblasts, comprising:
a) components for preparing a fully defined, preferably non self-degradable hydrogel matrix array having a stiffness in the range of 50 to 3000 Pa, preferably 50 to 2000 Pa and most preferably 50 to 1000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein the at least one crosslinking agent preferably comprises an enzymatically degradable motif, preferably a MMP-sensitive motif, and wherein at least one of the crosslinking agent and/or said optional bioactive agent comprise a RGD motif;
b) one or more different culture media, preferably comprising Wnt agonists such as R-spondin and Wnt 3a, preferably additionally FBS.

According to this embodiment, the present invention is also related to a kit of parts for testing the influence of drugs on cancer cells, preferably pancreatic ductal adenocarcinoma (PDAC) cells, that have been co-cultured with stromal cells, preferably fibroblasts, comprising:
a) components for preparing a fully defined, preferably non self-degradable hydrogel matrix array having a stiffness in the range of 50 to 3000 Pa, preferably 50 to 2000 Pa and most preferably 50 to 1000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS,
   at least one crosslinking agent, preferably a peptide comprising at least two, preferably two, cysteine moieties,
   optionally one or more biologically active molecules, wherein the at least one crosslinking agent preferably comprises an enzymatically degradable motif, preferably a MMP-sensitive motif, and wherein at least one of the crosslinking agent and/or said optional bioactive agent comprise a RGD motif;
b) one or more different culture media, preferably comprising Wnt agonists such as R-spondin and Wnt 3a, preferably additionally FBS
c) optionally, cells from a cell repository/biobank that have been created using the same extracellular matrix conditions.

The preselected extracellular matrix conditions of this embodiment can also be used in a method for testing the efficiency of therapies for a cancer patient, comprising the steps:
a) providing freshly isolated or frozen cancer cells, preferably pancreatic ductal adenocarcinoma cells, from a biopsy or a tissue resection of a cancer patient, and providing stromal cells isolated from patient, so as to establish a co-culture system;
b) establishing and expanding cells from said co-culture system, and applying one or more drugs to said cells by the method described above;
c) comparing the activity of the one or more drugs applied in step b) with the result of the treatment of said patient with one of said drugs applied in step b);
d) and/or providing drug activity results on patient organoids and corresponding genetic and phenotypic data of the disease to support physician in making decisions on how to treat the patient.

Patient biopsies or resections dedicated for the isolation of pancreatic ductal adenocarcinoma (PDAC) cells to establish organoids in step b), can be collected during a standard diagnostic procedure and subsequently transported to the site where step b) is to be conducted.

Step b) of this method is conducted as described above, i.e. by providing preselected extracellular matrix conditions comprising a fully defined, preferably non self-degradable hydrogel matrix array with discrete volumes having a stiffness in the range of 50 to 3000 Pa, preferably 50 to 2000 Pa and most preferably 50 to 1000 Pa and prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said cancer cells, preferably pancreatic ductal adenocarcinoma cells, co-cultured with stromal cells, preferably fibroblasts, so as to create fully defined three-dimensional extracellular matrix conditions that differ from each other in their biological, biophysical and/or biochemical characteristics;

allowing said cancer cells, preferably pancreatic ductal adenocarcinoma cells, and said stromal cells, preferably fibroblasts, to grow in said discrete volumes of said hydrogel matrix in the presence of one or more different culture media preferably comprising Wnt agonists such as R-spondin and Wnt 3a, preferably additionally FBS;

and adding one or more drugs to the cells grown in said discrete volumes of said hydrogel matrix;

wherein the at least one crosslinking agent preferably comprises an enzymatically degradable motif, preferably a MMP-sensitive motif, and wherein at least one of the crosslinking agent and/or said optional bioactive agent comprise a RGD motif.

The one or more drugs added to the cells grown in said discrete volumes of said hydrogel matrix comprise the one or more drugs used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests. Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Colorectal Cancer

Colorectal cancer (CRC) cells are known to exhibit heterogeneity (Roerink et al., (Nature, published online https://doi.org/10.1038/s41586-018-0024-3 (2018))). Significant differences in responses to drugs between closely related cells of the same tumor were observed. The same discussion as before for the pancreatic cells applies here.

With the present invention, it is possible to culture patient colorectal cancer (CRC) cells under conditions that sustain the growth and expansion of these cells. Subsequently, the cells are exposed to different drug treatments in order to select an efficient drug treatment for the cancer patient. Thus, the present invention provides a precision medicine platform enabling the growth and drug testing of CRC tissues in different microenvironments and therefore captures the specificities of multiple clones inside a single tumor.

According to the present invention, it could be shown that colorectal cancer (CRC) cells could be well cultured and tested using a combination of a fully defined soft or medium (50-2000 Pa stiffness) hydrogel matrix comprising at least one RGD adhesion motif and optionally laminin, preferably laminin-111 or laminin-511, and especially preferable natural mouse laminin-111 or recombinant human laminin-511, as an additional bioactive ligand, and a culture medium, preferably comprising Wnt agonists such as R-spondin and Wnt 3a.

Thus, according to this embodiment, the present invention is related to a method of testing the influence of drugs on colorectal cancer (CRC) cells, comprising the steps of:
a) providing preselected extracellular matrix conditions comprising a fully defined hydrogel matrix array with discrete volumes having a stiffness in the range of 50 to 2000 Pa and prepared by crosslinking, onto a substrate or into discrete volumes of a substrate, preferably a multi-well plate, different combinations of one or more different PEG hydrogel precursor molecules, in the presence of optionally one or more biologically active molecules comprising laminin, preferably laminin-111 or laminin-511, and especially preferable natural mouse laminin-111 or recombinant human laminin-511, at least one crosslinking agent, and said colorectal cancer cells, so as to create fully defined three-dimensional extracellular matrix conditions that differ from each other in their biological, biophysical and/or biochemical characteristics;
b) allowing said colorectal cancer cells to grow in said discrete volumes of said hydrogel matrix in the presence of one or more different culture media, preferably comprising Wnt agonists such as R-spondin and Wnt 3a;
c) adding one or more drugs to the cells grown in said discrete volumes of said hydrogel matrix;

wherein at least one of said crosslinking agent and/or said bioactive agent comprises a RGD motif.

Preferably, said PEG hydrogel precursor molecules are PEG-VS (Polyethylene glycol with terminal vinylsulfone moieties), especially preferable 4-arm or 8-arm PEG-VS, and/or PEG-Acr (Polyethylene glycol with terminal acrylate moieties), especially preferable 4-arm or 8-arm PEG-Acr.

More preferably, said fully defined self-degradable hydrogel matrix array is prepared by crosslinking a 50:50 mixture of PEG-VS, especially preferable 4-arm or 8-arm PEG-VS, and PEG-Acr, especially preferable 4-arm or 8-arm PEG-Acr, with a peptide comprising at least two, preferably two cysteine moieties as a crosslinking agent, wherein said crosslinking agent may comprise a RGD motif.

More preferably, said fully defined non self-degradable hydrogel matrix array is prepared by crosslinking PEG-VS, especially preferable 4-arm or 8-arm PEG-VS, with a peptide comprising at least two, preferably two cysteine moieties as a crosslinking agent, wherein said crosslinking agent may comprise a RGD motif.

As an optional bioactive ligand, a ligand comprising a bioactive motif including a RGD adhesion motif may be used. Examples of suitable RGD motifs are RGD, RGDS, RGDSP, RGDSPG, RGDSPK, RGDTP, RGDSPASSKP, PHSRNSGSGSGSGSGRGDSPG or any cyclic RGD motifs such as cyclo(RGDfC), but principally any known and successfully employed RGD sequences, in the field of hydrogels and cell culture, could be used.

Preferably, said optional bioactive ligand is selected from the group consisting of natural laminins, for example laminin-111, in particular mouse laminin-111, recombinant laminin isoforms such as recombinant human laminin-511, and biofunctional fragments thereof. Examples of suitable recombinant laminin isoforms are laminin-111, laminin-211, laminin-332, laminin-411, laminin-511, or laminin-521.

Preferably, said culture medium is characterized by the presence of Wnt agonists such as R-Spondin and Wnt 3a. According to a preferred embodiment, a culture medium may be used that is described in Vlachogiannis et al., Science 359, 920-926 (2018) (see e.g. supplementary material, p. 5, Human PDO culture media). Alternatively, the commercially available culture medium Intesticult® may be used. Especially preferred is the culture medium described in Vlachogiannis et al., which comprises Advanced DMEM/F12, supplemented with B27 additive, N2 additive, BSA, L-Glutamine, penicillin-Streptomycin, EGF, Noggin, R-Spondin 1, Gastrin, FGF-10, FGF-basic, Wnt-3A, Prostaglandin E 2, Y-27632, Nicotinamide, A83-01, SB202190 and optionally HGF.

Especially preferable, said colorectal cancer cells are from freshly isolated or frozen cells from a biopsy or tissue resection of a human or from patient-derived xenograft (PDX) tissue.

With said method, it is possible to grow, expand and subsequently test said colorectal cancer cells in a selected medium under conditions that recapitulate drug results observed in vivo.

According to an especially preferred embodiment, the extracellular matrix conditions are chosen such that the use of a naturally-derived matrix such as Matrigel® can be completely avoided.

According to this embodiment, the present invention is also related to a kit of parts for testing the influence of drugs on colorectal cancer cells, comprising:
a) components for preparing a fully defined hydrogel matrix array having a stiffness in the range of 50 to 2000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
   optionally one or more biologically active molecules comprising laminin, preferably laminin-111 or laminin-511, and especially preferable natural mouse laminin-111 or recombinant human laminin-511,
wherein at least one of said crosslinking agent and/or said bioactive agent comprises a RGD motif;
b) one or more different culture media, preferably comprising Wnt agonists such as R-spondin and Wnt 3a.

According to this embodiment, the present invention is also related to a kit of parts for testing the influence of drugs on colorectal cancer cells, comprising:
a) components for preparing a fully defined hydrogel matrix array having a stiffness in the range of 50 to 2000 Pa, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
   optionally one or more biologically active molecules comprising laminin, preferably laminin-111 or laminin-511, and especially preferable natural mouse laminin-111 or recombinant human laminin-511,
wherein at least one of said crosslinking agent and/or said bioactive agent comprises a RGD motif;
b) one or more different culture media, preferably comprising Wnt agonists such as R-spondin and Wnt 3a;
c) optionally, cells from a cell repository/biobank that have been created using the same extracellular matrix conditions.

The preselected extracellular matrix conditions of this embodiment can also be used in a method for testing the efficiency of therapies for a cancer patient, comprising the steps:
a) providing freshly isolated or frozen colorectal cancer cells from a biopsy or a tissue resection of a cancer patient;
b) establishing and expanding organoids from said cells, and applying one or more drugs to said organoids by the method described above;
c) comparing the activity of the one or more drugs applied in step b) with the result of the treatment of said patient with one of said drugs applied in step b);
d) and/or providing drug activity results on patient organoids and corresponding genetic and phenotypic data of the disease to support physician in making decisions on how to treat the patient.

Patient biopsies or resections dedicated for the isolation of colorectal cancer cells to establish organoids in step b), can be collected during a standard diagnostic procedure and subsequently transported to the site where step b) is to be conducted.

Step b) of this method is conducted as described above, i.e. by providing preselected extracellular matrix conditions comprising a fully defined hydrogel matrix array with discrete volumes having a stiffness in the range of 50 to 2000 Pa and prepared by crosslinking, onto a substrate or into discrete volumes of a substrate, preferably a multi-well plate, different combinations of one or more different PEG hydrogel precursor molecules in the presence of optionally one or more biologically active molecules comprising laminin, preferably laminin-111 or laminin-511, and especially preferable natural mouse laminin-111 or recombinant human laminin-511, at least one crosslinking agent, and said colorectal cancer cells, so as to create fully defined three-dimensional extracellular matrix conditions that differ from each other in their biological, biophysical and biochemical characteristics;

allowing said colorectal cancer cells to grow in said discrete volumes of said hydrogel matrix in the presence of one or more different culture media preferably comprising Wnt agonists such as R-spondin and Wnt 3a;

and adding one or more drugs to the cells grown in said discrete volumes of said hydrogel matrix;

wherein at least one of said crosslinking agent and/or said optional bioactive agent comprises a RGD motif.

The one or more drugs added to the cells grown in said discrete volumes of said hydrogel matrix comprise the drug(s) used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests.

Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Breast Cancer

There are distinct breast cancer subtypes, which require different culture conditions. The same discussion as before for the pancreatic cells applies here.

With the present invention, it is possible to culture breast cancer cells, for example, but not limited to the triple negative (TNBC) or HER2+ receptor status under conditions that sustain the growth and expansion of these cells. Subsequently, the cells are exposed to different drug treatments in order to select an efficient drug treatment for the cancer patient. Thus, the present invention provides a precision medicine platform enabling the growth and drug testing of breast cancer tissues in different microenvironments and therefore captures the specificities of multiple clones inside a single tumor.

According to the present invention, it could be shown that breast cancer cells could be well cultured and tested preferably using a fully defined enzymatic-degradable hydrogel matrix and preferably a culture medium comprising FBS (serum) or Wnt agonist such as R-spondin, preferably under hypoxic (low oxygen 5% O₂) conditions. For some subtypes (in particular TNBC subtype) at least one RGD adhesion motif and optionally laminin, preferably laminin-111 and especially preferably natural mouse laminin-111, as an additional bioactive ligand were preferable.

Thus, according to this embodiment, the present invention is related to a method of testing the influence of drugs on breast cancer cells, comprising the steps of:
a) providing preselected extracellular matrix conditions comprising a fully defined, preferably enzymatic-degradable hydrogel matrix array with discrete volumes and prepared by crosslinking, onto a substrate or into discrete volumes of a substrate, preferably a multi-well plate, different combinations of one or more different PEG hydrogel precursor molecules, in the presence of optionally one or more biologically active molecules, at least one crosslinking agent, and said breast cancer cells, so as to create fully defined three-dimensional extracellular matrix conditions that differ from each other in their biological, biophysical and/or biochemical characteristics;
b) allowing said breast cancer cells to grow in said discrete volumes of said hydrogel matrix in the presence of one or more different culture media, preferably comprising FBS (serum) or Wnt agonist such as R-spondin;
c) adding one or more drugs to the cells grown in said discrete volumes of said hydrogel matrix;

wherein said at least one crosslinking agent comprises preferably an enzymatically degradable motif, preferably a MMP-sensitive motif.

Preferably, said hydrogel matrix array has a soft or medium stiffness in the range of 50-2000 Pa.

Preferably, said PEG hydrogel precursor molecules are PEG-VS (Polyethylene glycol with terminal vinylsulfone moieties), especially preferable 4-arm or 8-arm PEG-VS, and/or PEG-Acr (Polyethylene glycol with terminal acrylate moieties), especially preferable 4-arm or 8-arm PEG-Acr.

More preferably, said fully defined hydrogel matrix array is prepared by crosslinking PEG-VS, especially preferable 4-arm or 8-arm PEG-VS, or a 50:50 mixture of PEG-VS, especially preferable 4-arm or 8-arm PEG-VS, and PEG-Acr, especially preferable 4-arm or 8-arm PEG-Acr, with a peptide comprising at least two, preferably two cysteine moieties as a crosslinking agent, wherein said crosslinking agent may comprise an enzymatically degradable motif, preferably a MMP-sensitive motif.

As an optional bioactive ligand, a ligand comprising a bioactive motif including a RGD adhesion motif may be used. Examples of suitable RGD motifs are RGD, RGDS, RGDSP, RGDSPG, RGDSPK, RGDTP, RGDSPASSKP, PHSRNSGSGSGSGSGRGDSPG or any cyclic RGD motifs such as cyclo(RGDfC), but principally any known and successfully employed RGD sequences, in the field of hydrogels and cell culture, could be used.

As an optional bioactive ligand, a ligand comprising a bioactive motif may be used. Preferably, said optional bioactive ligand is selected from the group consisting of natural laminins, for example laminin-111, in particular mouse laminin-111, recombinant laminin isoforms, and biofunctional fragments thereof. Examples of suitable recombinant laminin isoforms are laminin-111, laminin-211, laminin-332, laminin-411, laminin-511, or laminin-521.

As an optional bioactive ligand, a ligand comprising glycosaminoglycans such as hyaluronic acid and hyaluronan may be used. Examples of hyaluronic acid are hyaluronic acid 50k, hyaluronic acid 1000k, hyaluronate thiol 50k or hyaluronate thiol 1000k.

According to a preferred embodiment, a culture medium may be used that is described in Sachs et al. (Cell 172 2018, 1-14 (see e.g. supplementary material, table S2)). Alternatively, the commercially available culture media Intesticult™, Mammocult™, WITP™, MEBM™, or StemPro™ hESC SFM may be used. Especially preferred is the culture medium described in Sachs et al., which comprises R-Spondin 1 conditioned medium or R-Spondin 3, Neuregulin 1, FGF 7, FGF 10, EGF, Noggin, A83-01, Y-27632, SB202190, B27 supplement, N-Acetylcysteine, Nicotinamide, GlutaMax 100x, Hepes, Penicillin/Streptomycin, Primocin and Advanced DMEM/F12. Other media such as IMDM+FBS (serum), or those described in Liu et al. (Sci Rep 2019, (9):622), or Lancaster et al. (Nat Biotechnol 2017 35(7): 659-666) may be used.

According to a preferred embodiment, hypoxic (low oxygen 5% O₂) conditions are preferred.

Especially preferable, said breast cancer cells are from freshly isolated or frozen cells from a biopsy or tissue resection of a human or from patient-derived xenograft (PDX) tissue.

With said method, it is possible to grow, expand and subsequently test said breast cancer cells in a selected medium under conditions that recapitulate drug results observed in vivo.

According to an especially preferred embodiment, the extracellular matrix conditions are chosen such that the use of a naturally-derived matrix such as Matrigel® can be completely avoided.

According to this embodiment, the present invention is also related to a kit of parts for testing the influence of drugs on breast cancer cells, comprising:
a) components for preparing a fully defined, preferably enzyuratic-degradable hydrogel matrix array, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
   optionally one or more biologically active molecules, wherein said at least one crosslinking agent preferably comprises an enzymatically degradable motif, preferably a MMP-sensitive motif;
b) one or more different culture media, preferably comprising FBS (serum) or Wnt agonist such as R-spondin.

According to this embodiment, the present invention is also related to a kit of parts for testing the influence of drugs on breast cancer cells, comprising:
a) components for preparing a fully defined, preferably enzymatic-degradable hydrogel matrix array, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
   optionally one or more biologically active molecules, wherein said at least one crosslinking agent preferably comprises an enzymatically degradable motif, preferably a MMP-sensitive motif;
b) one or more different culture media, preferably comprising FBS (serum) or Wnt agonist such as R-spondin;
c) optionally, cells from a cell repository/biobank that have been created using the same extracellular matrix conditions.

The preselected extracellular matrix conditions of this embodiment can also be used in a method for testing the efficiency of therapies for a cancer patient, comprising the steps:
a) providing freshly isolated or frozen breast cancer cells from a biopsy or a tissue resection of a cancer patient;
b) establishing and expanding organoids from said cells, and applying one or more drugs to said organoids by the method described above;
c) comparing the activity of the one or more drugs applied in step b) with the result of the treatment of said patient with one of said drugs applied in step b).
d) and/or providing drug activity results on patient organoids and corresponding genetic and phenotypic data of the disease to support a physician in making decisions on how to treat the patient.

Patient biopsies or resections dedicated for the isolation of breast cancer cells to establish organoids in step b), can be collected during a standard diagnostic procedure and subsequently transported to the site where step b) is to be conducted.

Step b) of this method is conducted as described above, i.e. by providing preselected extracellular matrix conditions comprising a fully defined, preferably enzymatic-degradable hydrogel matrix array with discrete volumes prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said breast cancer cells, so as to create fully defined three-dimensional extracellular matrix conditions that differ from each other in their biological, biophysical and biochemical characteristics;

allowing said breast cancer cells to grow in said discrete volumes of said hydrogel matrix in the presence of one or more different culture media preferably comprising FBS (serum) or Wnt agonist such as R-spondin;

and adding one or more drugs to the cells grown in said discrete volumes of said hydrogel matrix;

wherein at least one of said crosslinking agent preferably comprises an enzymatically degradable motif, preferably a MMP-sensitive motif.

The one or more drugs added to the cells grown in said discrete volumes of said hydrogel matrix comprise the drug(s) used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests.

Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Prostate Cancer

This embodiment shows the benefits provided by the present invention with respect to the problem of selected growth of cancer cells over normal cells (e.g. wild-type healthy cells, stromal cells).

For some organs, patient-derived cancer cells that form tumor organoids tend to grow ex vivo more slowly than their healthy (wild-type) counterparts or associated stromal cells, and/or currently used ex vivo conditions based on Matrigel® or equivalent matrices are not able to select and favour the growth of one tissue type vs. the other.

Therefore, normal cells tend to overgrow the cancer organoids cultures, unless specific measures are taken. Despite the fact that in some cases modifications of the media composition could solve this issue, the overgrowth of normal cells (healthy cells) vs. cancer cells still remains a problem, for example for prostate cancer. This impairs the establishment of ex vivo growth of patient-derived cancer cells as physiological preclinical model e.g. to determine which drug or drug combination may work to treat the specific patient.

Overgrowth of normal cells has been particularly observed in the case of prostate cancer organoids (Drost et al., Development (2017) 144, 968-975). For example, less than ten prostate cancer cell lines currently exist (whereas for Colon cancer more than 50 cell lines exist), and none of them adequately reflects the correct cancer disease (ATCC and ECACC cell banks). Therefore, the ability to grow e.g. prostate cancer organoids while impeding the growth of normal cells, would significantly impact the development of new drugs by using more physiological preclinical models and enable the use of patient-specific cell models for personalized medicine applications.

Drost et al., Development (2017) 144, 968-975 (see pages 971-972 “Personalized cancer therapy”) summarizes how the selection between (i) cancer cells and (ii) normal cells (wild-type) is done to grow ex vivo pure cell populations of (i) vs. (ii). Briefly, where it is possible, this is achieved by adding or omitting chemicals/growth factors in cell culture Media. However, for prostate cancer this is not as easy, for example, as for certain colon cancers that harbour specific genetic mutations, which make their successful culture independent of certain chemicals in contrast to their wild-type counterparts. As described in Drost et al., Nature protocols, Vol. 11 no.2 (2016) 347 (see pages 347-348 “Limitation of the method”), their culture protocol, was not good enough for growing organoids derived from primary prostate cancers, most likely due to the fact that ex vivo tumor cells do not have a selective advantage over normal cells. Consequently, the normal prostate cells that are normally present within each cancer sample tissues, seem to overgrow the tumor cells (See also Karthaus et al., Cell 159, 163-175, Sep. 25, 2014, on page 171 last “Discussion” paragraph). Furthermore, a similar problem was also observed with sample biopsies from prostate metastasis in bone and soft tissues (Gao et al., Cell 159, 176-187, Sep. 25, 2014, see page 177-178 “Results”), where normal host tissue cells (e.g. stroma and/or epithelial cells) were overtaking the cancer cells.

Other examples of normal cell overgrowth that are less commonly reported include breast cancer and lung cancer ex vivo cultures. In a study published by Sachs et al. (Cell 172 2018, 1-14, demonstrating the establishment of >100 primary and metastatic breast cancer organoids, there are a couple of instances in which the pathology of the organoid is classified as normal while the original tissue pathology was classified as tumor (Sachs et al., Cell 172 2018, 1-14, Table S3). This can also be observed in lung cancer organoids, for example, derived from patients harbouring a mutation in p53, in which normal versus cancerous cells can be selected by adding chemicals to the cell culture Media (Sachs et al., The EMBO Journal e 100300|2019). However, if there is no p53 mutation, there is no way to prevent normal cell overgrowth.

According to this embodiment of the present invention, preselected extracellular matrix conditions were identified that promote the growth of prostate cancer cells while at the same time impeding the establishment of their normal counterpart. This allows the establishment of a screening method where cancer cells can be reliably evaluated that otherwise would be overgrown by their normal counterparts.

In detail, it was found that normal prostate cells are growing only in gel formulations containing a RGD adhesion motif, and their growth is better in soft gels compared to medium or hard ones. On the other hand, it was found that prostate cancer cells isolated from patient or from patient-derived xenograft (PDX) tumors show a similar growth in gels with and without the presence of a RGD motif. Some prostate cancer cells isolated from patient-derived xenograft (PDX) tumors grow even better in gel without RGD (soft or medium stiffness).

Thus, according to this embodiment, the present invention is related to a method of testing the influence of drugs on cancer cells that grow ex vivo more slowly than their normal counterparts or associated stromal cells, preferably prostate cancer cells, comprising the steps of:
a) providing preselected extracellular matrix conditions comprising a fully defined hydrogel matrix array with discrete volumes, prepared by crosslinking, onto a substrate or into discrete volumes of a substrate, preferably a multi-well plate, different combinations of one or more different PEG hydrogel precursor molecules, in the presence of optionally one or more biologically active molecules, at least one crosslinking agent, and said cancer cells, so as to create fully defined three-dimensional extracellular matrix conditions that differ from each other in their biological, biophysical and/or biochemical characteristics;
b) allowing said cancer cells to grow in said discrete volumes of said hydrogel matrix in the presence of one or more different culture media;
c) adding one or more drugs to the cells grown in said discrete volumes of said hydrogel matrix;

wherein said crosslinking agent and said optional bioactive agent do not comprise any RGD motif.

Preferably, said PEG hydrogel precursor molecules are PEG-VS (Polyethylene glycol with terminal vinylsulfone moieties), especially preferable 4-arm or 8-arm PEG-VS, and/or PEG-Acr (Polyethylene glycol with terminal acrylate moieties), especially preferable 4-arm or 8-arm PEG-Acr.

More preferably, said fully defined self-degradable hydrogel matrix array is prepared by crosslinking PEG-Acr, especially preferable 4-arm or 8-arm PEG-Acr, or a 50:50 mixture of PEG-VS, especially preferable 4-arm or 8-arm PEG-VS, and PEG-Acr, especially preferable 4-arm or 8-arm PEG-Acr, with a peptide comprising at least two, preferably two cysteine moieties as a crosslinking agent.

Preferably, said hydrogel matrix array has a soft or medium stiffness in the range of 50-2000 Pa.

According to another preferred embodiment, said fully defined, non self-degradable hydrogel matrix array is prepared by crosslinking PEG-VS, especially preferable 4-arm or 8-arm PEG-VS, with a peptide comprising at least two, preferably two cysteine moieties as a crosslinking agent.

As an optional bioactive ligand, a ligand comprising a bioactive motif may be used. Preferably, said optional bioactive ligand is selected from the group consisting of Tenascin C and Glypican, natural laminins, for example laminin-111, in particular mouse laminin-111, recombinant laminin isoforms, and biofunctional fragments thereof. Examples of suitable recombinant laminin isoforms are laminin-111, laminin-211, laminin-332, laminin-411, laminin-511 or laminin-521.

Preferably, culture media were pre-selected to grow prostate cancer cells. Commercially available culture media, e.g. Mammocult™, WIT-P™, StemPro™ hESC SFM, PrEGM™ BulletKit™ from Lonza (ref. CC-3166), and NutriStem® hPSC XF may be used, as well as media as described in WO 2015/173425 A1 or Drost et al. (Nature Protocol 11, 347-358, January 2016) or Beshiri et al. (Clinical Cancer Research 24, 4332-4345), May 2018) or Puca et al. (Nature Communications 9:2404, 1-10, June 2018) or in Ince et al. (Cancer Cell 12, 160-170, August 2007), are suitable for the growth of cancer cells. It was found that the culture medium described in WO 2015/173425 A1 favours growth of cancer cells. Especially preferred is therefore a culture medium which comprises Glutamine, BSA, Transferrin, Noggin, FGF (2 or basic), FGF 10, EGF, R-Spondin conditioned medium or recombinant, Penicillin/Streptomycin, Glutathione, Nicotinamide, DHT, Prostaglandin E2, A83-01, Y-27632, N-acetylcysteine, SB202190 and Hepes.

Especially preferable, said cancer cells are from freshly isolated or frozen cells from a biopsy or tissue resection of a human or from patient-derived xenograft (PDX) tissue.

With said method, it is possible to grow and subsequently test said cancer cells in a selected medium under conditions that recapitulate drug results observed in vivo, without being overgrown by their normal counterparts or associated stromal cells.

According to an especially preferred embodiment, the extracellular matrix conditions are chosen such that the use of a naturally-derived matrix such as Matrigel® can be completely avoided.

According to this embodiment, the present invention is also related to a kit of parts for testing the influence of drugs on cancer cells that grow ex vivo more slowly than their normal counterparts or associated stromal cells, preferably prostate cancer cells, comprising:
a) components for preparing a fully defined hydrogel matrix array, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
   optionally one or more biologically active molecules, wherein said crosslinking agent and said optional bioactive agent do not comprise any RGD motif;
b) one or more different culture media.

According to this embodiment, the present invention is also related to a kit of parts for testing the influence of drugs on cancer cells that grow ex vivo more slowly than their normal counterparts or associated stromal cells, preferably prostate cancer cells, comprising:
a) components for preparing a fully defined hydrogel matrix array, so as to create fully defined three-dimensional extracellular matrix conditions that differ from each other in their biological, biophysical and/or biochemical characteristics, said components comprising
   one or more different PEG hydrogel precursor molecules, preferably PEG-VS, especially preferable 4-arm or 8-arm PEG-VS, and/or PEG-Acr, especially preferable 4-arm or 8-arm PEG-Acr,
   at least one crosslinking agent, preferably a peptide comprising at least two, preferably two, cysteine moieties,
optionally one or more biologically active molecules, wherein said crosslinking agent and said optional bioactive agent do not comprise any RGD motif;
b) one or more different culture media;
c) optionally, cells from a cell repository/biobank that have been created using the same extracellular matrix conditions.

The preselected extracellular matrix conditions of this embodiment can also be used in a method for testing the efficiency of therapies for a cancer patient, comprising the steps:
a) providing freshly isolated or frozen cancer cells from a biopsy or a tissue resection of a cancer patient;
b) establishing and expanding organoids from said cells, and applying one or more drugs to said organoids by the method described above;
c) comparing the activity of the one or more drugs applied in step b) with the result of the treatment of said patient with one of said drugs applied in step b);
d) and/or providing drug activity results on patient organoids and corresponding genetic and phenotypic data of the disease to support physician in making decisions on how to treat the patient.

Patient biopsies or resections dedicated for the isolation of prostate cancer cells to establish organoids in step b), can be collected during a standard surgery or diagnostic procedure and subsequently transported to the site where step b) is to be conducted.

Step b) of this method is conducted as described above, i.e. by providing preselected extracellular matrix conditions comprising a fully defined hydrogel matrix array with discrete volumes, prepared by crosslinking, onto a substrate or into discrete volumes of a substrate, preferably a multi-well plate, different combinations of one or more different PEG hydrogel precursor molecules in the presence of optionally one or more biologically active molecules, at least one crosslinking agent, and said cancer cells, so as to create fully defined three-dimensional extracellular matrix conditions that differ from each other in their biological, biophysical and biochemical characteristics;

allowing said cancer cells to grow in said discrete volumes of said hydrogel matrix in the presence of one or more different culture media;

and adding one or more drugs to the cells grown in said discrete volumes of said hydrogel matrix;

wherein said crosslinking agent and said optional bioactive agent do not comprise any RGD motif.

The one or more drugs added to the cells grown in said discrete volumes of said hydrogel matrix comprise the drug(s) used for anticancer standard of care (SoC) treatment of the patient.

According to a preferred embodiment, the biopsy or resection can additionally be processed for histological analysis and/or omics testing (e.g. NGS) to establish the baseline for reference. The results of these additional analyses can also be used for comparing and/or correlating the above ex vivo and in vivo tests.

Based on this method, it is possible to reliably assess whether the applied anticancer standard of care (SoC) treatment is suitable, or whether a different drug treatment regime tested ex vivo as described above might be more promising. Thus, with the present invention the cancer treatment can be personalized and optimized. Functional in vitro data can be generated that may increase accuracy of treatment decisions by health care providers.

### Dynamic Organoid Growth Image Analysis Method

There is a need to quantify accurately and easily different parameters of organoid growth (e.g. growth rate, organoid number, organoid size). As for conventional 2D cell culture, quantification of organoid growth can be achieved indirectly by using fluorometric, colorimetric or luminescent methods which measure the quantities of metabolites in culture wells (e.g. Alamar Blue, MTT, Cell Titer Glow 3D). All these indirect assays, even when they are not lethal for the cells, can affect the fitness of the organoids and totally prevent the possibility to further use the grown organoids (e.g. for drug testing, regenerative medicine).

Using non-invasive (and label-free) methods as light-microscopy to quantify organoid growth is therefore a favoured alternative in long-term culture and/or when the organoids need to be kept as “native” & “untouched” as possible (e.g. for regenerative medicine, biobanking). The need for quantification of high throughput imaging of 3D organoid cultures has led to the adaptation of 2D methods (Carpenter et al., Genome Biology 2006, 7:R100) or the development of new automatized detection and image segmentation algorithms which enable the counting and measurement of organoid in a fast, reproducible and unbiased manner. These methods are more easily and more accurately performed using fluorescent markers (Robinson et al., PLoSONE 10(12): e0143798. doi:10.1371/journal.pone.01437982015, Boutin et al., Nature scientific reports (2018)8:11135, DOI:10.1038/s41598-018-29169-0 2018), which, again is not compatible with the use of “untouched” & “label-free” patient-derived cells or re-implantation protocols, as they require immunofluorescence staining or fluorescent transgene expression.

Recently, in Borten et al., Nature scientific reports (2018) 8:5319, DOI:10.1038/s41598-017-18815-8 2018, a Matlab (from Mathworks company) based algorithm called OrganoSeg was developed to specifically analyse organoids from 3D brightfield images, thereby allowing to detect, segment (i.e. partitioning a digital image into specific set of pixels) and quantify many parameters from living native organoids grown in 3D (Borten 2018). This open-source software allows for identification and multiparametric morphometric classification of organoids based on size, sphericity and shape of the detected features at a given timepoint.

However, despite being accurate and powerful, this tool does not allow taking into account the time dimension and would require multiple analyses at different timepoints, and thus consequent compilation work, to properly assess the dynamics of organoid growth.

According to the present invention, a new analysis method is provided. Based on a MATLAB code a new method was developed, which is able to align brightfield images acquired at different timepoints and automatically identify and segment organoids based on their intensity. This program uses the same method as OrganoSeg to segment objects from Brightfield images. The main difference resides in the use made of these segmented objects: While OrganoSeg uses the size and morphologies to classify different types of organoids at a given discrete timepoint, this new program allows for the dynamic follow up of organoid growth in one single analysis and thus the calculation of OFE/AIF and drug response. Accordingly, the program provides the following dynamic information about the organoid growth:
Organoid Forming Efficiency (OFE) and,
Area Increase Factor (AIF).

With the method of the present invention, it is also possible to generate aligned time-lapse videos for each well acquired, and “Time Projections” representing the overall growth of organoids along the culture duration in a single rendered image. Those time projections are easy to include in presentations and publications.

When single cells or small clusters of cells are encapsulated in 3D extracellular matrix, only a subset of these cells is able to grow and generate organoids; this is what is designed as the “Organoid Forming Efficiency” (OFE) of the culture. With the method of the present invention, the number of encapsulated cells at Day 0 is quantified. Moreover, the method allows the user to define a threshold for the size of what is considered to be an organoid. It then provides the OFE for any particular timepoint in the assay.

While the OFE gives an indication of the percentage of cells in the original culture capable of developing in organoids (e.g. stem cells), with the method of the present invention also the growth rate of the overall organoid culture is quantified by calculating the increase in area along time after segmentation of the time-lapse. This is the “Area Increase Factor” (AIF), which is corresponding to the ratio of the total area occupied by single cells at Day 0 to the total area occupied by the organoids at any given day. The method of the present invention allows for the selection of initial and final days to calculate the AIF.

Combining the OFE and AIF scores gives useful information on the fitness and performance of a given extracellular matrix condition.

This semi-automatized image analysis method according to the present invention allows for the temporal investigation and analysis of organoid growth in high throughput set-ups. It provides unbiased and reproducible scoring reflecting the fitness and performance of extracellular matrix conditions for any organoid cultures without the need of markers and/or detrimental assays. It also allows for semi-automatic quantification of patient-derived organoids drug test results (e.g. IC₅₀-value determination).

### Example 1: Testing of Lung Cancer Cells

### Example 1a: Lung Cancer Cells Overexpressing the c-Met Receptor

From a patient, lung cancer cells overexpressing the c-Met receptor were obtained from PDX cells. Upon activation through ligand binding, the c-Met receptor autophosphorylates and activates several signaling cascades within the cell.

Treatment of patient-derived xenograft (PDX) cells of non-small cell lung cancer (NSCLC) model LXFA-1647 with a drug targeted against c-Met (c-Met inhibitor: PF-04217903, Selleck Chemicals) inhibits its autophosphorylation and induces tumor growth regression in vivo.

A PEG was used as a hydrogel precursor molecule for making a non self-degradable hydrogel. As a crosslinking agent, peptides containing at least two, preferably two cysteine moieties were used which varied in their amino acid sequence, in particular with respect to the presence or absence of a RGD adhesion motif and with respect to the presence or absence of a MMP degradation sequence. A further variation that was made to some hydrogels was the attachment of a bioactive ligand comprising a RGD adhesion motif or/and a ligand selected from the group consisting of natural laminins, recombinant laminin isoforms, and biofunctional fragments thereof. Examples of suitable recombinant laminin isoforms are laminin-111, laminin-211, laminin-332, laminin-411, laminin-511, or laminin-521. An array of hydrogels varying in the above preselected features was established, by the method described above.

The mechanical properties of the hydrogels were also varied (soft (50-1000 Pa), medium (1000-2000 Pa) or hard (2000-3000 Pa) gels).

For comparison, tests were also conducted in the undefined natural-derived matrix Matrigel®.

The culture medium was preselected to comprise the above described c-Met inhibitor PF-04217903 (Selleck Chemicals), i.e. a drug targeting c-met and inhibiting its autophosphorylation, or a drug used in standard of care (SoC) treatment of this cancer type (docetaxel). The respective drug was added to the culture media after 1 to 8 days of culture (1 to 8 days post cell encapsulation). The drug response was measured after 5 to 10 days post-drug addition. As a preferred culture medium, a medium was preselected that is characterized by the presence of FBS (serum) or Wnt agonists such as R-spondin. According to this example, a culture medium was used that was adapted from the medium described in Sachs et al. (The EMBO Journal e 100300|2019). The preferred culture medium comprised AdDMEM/F12 medium supplemented with glutamine, Noggin, EGF, fibroblast growth factor 7 and 10 [FGF7 and FGF10], HGF, R-spondin-conditioned medium, Primocin, penicillin/streptomycin, N-acetyl-L-cysteine, Nicotinamide, A83-01, SB202190 (p38-inhibitor), Y-27632 (rock inhibitor), B27 supplement and HEPES. Target expression (c-Met and Phospho c-Met) was detected by Western-blot in corresponding growth conditions.

The results are shown in FIGS. 1a, 1b and 1c.

With these preselected conditions, ex vivo growth (ex vivo culture, extracellular matrix) conditions that recapitulate drug results observed in vivo (i.e. activity of the c-Met inhibitor PF-04217903 (Selleck Chemicals)) were identified. These extracellular matrix conditions were qualified as “responder conditions”.

In this example, the most preferred responder conditions were the use of a non self-degradable hydrogel made from a respective PEG hydrogel precursor molecules and, as a crosslinking agent, a peptide containing two cysteine moieties without any RGD motif (either in the crosslinking agent or attached to the hydrogel). Said hydrogel has a soft stiffness in the range of 50-1000 Pa (example 1a), even more preferably 250-500 Pa.

In the same assay, other conditions were identified that result in drug resistance. Drug resistance was found to be dependent on the microenvironment, i.e. extracellular matrix or soluble factors. In particular, it could be shown that upon the attachment of 1 mM of a bioactive ligand comprising a RGD motif to the hydrogel, the tumor cells became resistant to the above described c-Met inhibitor PF-04217903 (Selleck Chemicals). Those conditions are qualified as “non-responder conditions” (example 1b).

Finally, it could be shown in the same assay that the use of Matrigel® as matrix also provided “non-responder conditions” (comparative example 1) in which the tumor cells did not respond to the above described c-Met inhibitor PF-04217903 (Selleck Chemicals).

In FIG. 1b, the effect of a standard of care (SoC) treatment with Docetaxel as well as the effect of treatment with the c-met-inhibitor PF-04217903 (Selleck Chemicals) under the conditions of example 1a are shown. Both drugs were clearly effective.

In contrast thereto, in FIG. 1c it is shown that under the conditions of comparative example 1 (Matrigel®), only an effect of a standard of care (SoC) treatment with Docetaxel could be observed. No effect of treatment with the c-met-inhibitor PF-04217903 (Selleck Chemicals) was observable. Accordingly, FIGS. 1a-1c) show that only under the preselection conditions of the present invention an effect of a c-met-inhibitor on the examined cells could be seen. When working under conventional conditions (i.e. using Matrigel®), the possible treatment with a c-met inhibitor would not have been recognized.

### Example 1b: Lung Cancer Cells Overexpressing the EGFR Receptor

Example 1a was repeated with lung cancer cells overexpressing the EGFR receptor. These cells were obtained from PDX cells.

Using the “responder conditions” of example 1a (i.e. without any RGD motif (either in the crosslinking agent or attached to the hydrogel)), in example 1b no effect of treatment with a c-met inhibitor could be observed, as was expected due to the lack of autophosphorylation of the c-met receptor in the cells tested in example 1b (see FIG. 1d). On the other hand, the EGFR receptor as well as its phosphorylated form were overexpressed under these conditions (FIG. 1d), and drugs acting on the EGFR receptor (Erlotinib and Cetuximab) showed a clear effect (similar to conditions of comparative example 1 using Matrigel® (data not shown)), as well as the SoC treatment with Paclitaxel (FIG. 1e).

### Example 2: Testing of Pancreatic Cancer Cells

Pancreatic ductal adenocarcinoma (PDAC) cancer cells from a patient were first expanded in mice as a PDX model. The PDX-derived cells were grown in a range of extracellular matrix conditions.

Treatment of patient-derived xenograft (PDX) cells of pancreatic ductal adenocarcinoma (PDAC) cancer model PAXF736 with a drug targeted against EGFR (EGFR inhibitor: Cetuximab) reduces the tumor growth in vivo.

A PEG was used as a hydrogel precursor molecule for making a non self-degradable hydrogel. As a crosslinking agent, peptides containing at least two, preferably two cysteine moieties were used which varied in their amino acid sequence, in particular with respect to the presence or absence of a RGD adhesion motif and with respect to the presence or absence of a MMP degradation sequence. A further variation that was made to some hydrogels was the attachment of a bioactive ligand comprising a RGD or a cyclic RGD adhesion motif, or alternatively a bioactive ligand comprising a DGEA motif. An array of hydrogels varying in the above preselected features was established, by the method described above.

The mechanical properties of the hydrogels were also varied (hard (2000-3000 Pa), medium (1000-2000 Pa) or soft (50-1000 Pa) gels).

A variety of different known, commonly employed and/or commercially available culture media was used.

In FIG. 2a and FIG. 2b, the results are shown for a soft non self-degradable PEG hydrogel with a crosslinking moiety without RGD motif, and with a bioactive ligand comprising a RGD adhesion motif (example 2a), as well as for a soft non self-degradable PEG hydrogel with a crosslinking moiety with RGD motif, and with a bioactive ligand comprising a DGEA adhesion motif (example 2b). For comparison, tests were also conducted in the undefined natural-derived matrix Matrigel® (comparative example 2).

It can be seen from FIG. 2a that all tested hydrogels led to a comparable growth of patient-derived xenograft (PDX) cells of pancreatic ductal adenocarcinoma (PDAC) cancer model PAXF736.

It can be seen from FIG. 2b that the hydrogel according to example 2a showed a drug sensitivity (for Cetuximab) comparable to that of Matrigel® (comparative example 2). On the other hand, the hydrogel according to example 2b showed a much higher drug sensitivity.

In FIG. 2c, it can be seen that when using a soft gel (50-1000 Pa, examples 2c and 2d) or medium gel (1000-2000 Pa, examples 2e and 2f) in the presence of a RGD motif and in the presence (examples 2c and 2e) or absence (examples 2d and 2f) of a MMP-sensitive motif, a very good growth of PDAC cells could be achieved, which was comparable to the growth of PDAC cells in Matrigel® (comparative example 2).

It was found that the presence of Wnt agonists such as R-spondin and Wnt 3a in the culture medium was important for cell growth. Also it was found that the hydrogel matrix should comprise at least one RGD motif.

This example shows the advantage of preselection according to the present invention. When working under conventional extracellular matrix conditions using Matrigel® (comparative example 2), no effect of an EGFR inhibitor on the tested cells was observable. Accordingly, a possibly effective treatment of this cancer type would not have been identified.

A comparison of examples 2a and 2b shows another advantage of the preselection according to the present invention. By using different preselection conditions that are principally suitable for a specific cell type (here presence of a RGD motif), it is possible to identify a possible resistance of the tested cells. In example 2a, the observed drug sensitivity against the EGFR inhibitor Cetuximab was much lower as compared to example 2b, indicating that treatment of this specific cancer cell type with an EGFR inhibitor alone might not be sufficient.

### Example 3: Testing of Pancreatic Cancer Cells Co-Cultured with Fibroblasts

In a further experiment, co-culturing of PDAC cells (from PDO pre-established in Matrigel®) with different ratios of cancer associated fibroblasts (isolated from patient and pre-expanded in 2D culture) was examined in a range of extracellular matrix conditions. The fibroblasts in the co-culture were identified using a specific marker (CD90).

In FIG. 3, the results of co-culturing 33% PDAC cells with 67% fibroblasts are shown in a hydrogel comprising both a RGD motif and an enzymatically (MMP) degradable moiety (example 3a), in a hydrogel comprising only a RGD motif and no enzymatically degradable moiety (example 3b), in a hydrogel comprising no RGD motif and only an enzymatically degradable moiety (example 3c), and in a hydrogel comprising no RGD motif and no enzymatically degradable moiety (example 3d). For comparison, the results with the conventional undefined natural-derived matrix Matrigel® (comparative example 3) are shown.

It can be seen that the best co-culturing results were obtained in example 3a, i.e. in a preferably soft PEG hydrogel comprising both a RGD motif and an enzymatically degradable moiety.

This example shows that it is possible to preselect conditions depending on whether simultaneous growth of other cells such as fibroblasts should be permitted or not.

### Example 4: Testing of Colorectal Cancer Cells

Colorectal cancer (CRC) cells from a patient and pre-established in Matrigel® were grown in a range of extracellular matrix conditions.

PEG was used as a hydrogel precursor molecule to provide a non self-degradable PEG hydrogel, or alternatively a 50:50 mixture of a non self-degradable PEG hydrogel and a self-degradable PEG hydrogel. As a crosslinking agent, peptides containing at least two, preferably two cysteine moieties were used which varied in their amino acid sequence, in particular with respect to the presence or absence of a RGD adhesion motif and with respect to the presence or absence of a MMP degradation sequence. A further variation that was made to some hydrogels was the attachment of a bioactive ligand comprising a RGD adhesion motif, or alternatively of a ligand selected from the group consisting of natural laminins, for example laminin-111, recombinant laminin isoforms such as recombinant human laminin-511, and biofunctional fragments thereof. Examples of suitable recombinant laminin isoforms are laminin-111, laminin-211, laminin-332, laminin-411, laminin-511, or laminin-521. An array of hydrogels varying in the above preselected features was established, by the method described above.

The mechanical properties of the hydrogels were also varied (hard (2000-3000 Pa), medium (1000-2000 Pa) or soft (50-1000 Pa) gels).

A variety of different known, commonly employed and/or commercially available culture media was used.

The results are shown in FIG. 4. FIG. 4 provides Brightfield images of human colon cancer organoids grown for 0 and 11 days.

In examples 4a to 4c hydrogels were used that were non self-degradable and non-enzymatically degradable. In example 4b, the crosslinking moiety comprised a RGD motif, wherein in examples 4a and 4c a bioactive ligand comprising a RGD motif was attached in a dangling manner. In example 4b, a bioactive ligand was attacked in a dangling manner that was laminin-111. The hydrogels according to examples 4a and 4b were soft (below 500 Pa), whereas the hydrogel according to example 4c was medium (above 1000 Pa). In example 4d, a hydrogel was used that was self-degradable, but non-enzymatically degradable, and had an initial stiffness in the range from 400 to 600 Pa. Said hydrogel had a crosslinking moiety that comprised a RGD motif, and laminin-111 as a bioactive ligand. For comparison, tests were also conducted in the undefined natural-derived matrix Matrigel® (comparative example 4).

It was shown that in the hydrogels according to examples 4a to 4d cell growth comparable to the standard Matrigel® was obtained, but in defined conditions (unlike Matrigel®).

On the other hand, in example 4e a hydrogel was used that was non self-degradable, but enzymatically degradable and furthermore did not comprise any RGD motif. Under these conditions, the tested CRC cells did not grow.

In example 4f, a self-degradable hydrogel with an initial stiffness around 400-600 Pa, an RGD motif (incorporated in the crosslinker) and recombinant human laminin-511 as bioactive agent was used. Very good growth of the tested CRC cells was observed.

It was found that the presence of Wnt agonists such as R-spondin and Wnt 3a in the culture medium was favorable for cell growth. Also it was found that the hydrogel matrix should comprise at least one RGD motif and optionally at least one bioactive ligand selected from the group consisting of natural laminins, for example laminin-111, recombinant laminin isoforms such as recombinant human laminin-511, and biofunctional fragments thereof.

### Example 5: Testing of Breast Cancer Cells

Breast cancer cells derived from patients with distinct cancer subtypes (Triple Negative (TNBC) or HER2+ receptor status) were first expanded in mice as PDX models. The PDX-derived cells were then grown in a range of extracellular matrix conditions.

A PEG was used as a hydrogel precursor molecule to provide a non self-degradable hydrogel. As a crosslinking agent, peptides containing at least two, preferably two cysteine moieties were used which varied in their amino acid sequence, in particular with respect to the presence or absence of a RGD adhesion motif and the presence or absence of a MMP-sensitive motif.

A further variation that was made to some hydrogels was the attachment of a bioactive ligand comprising a RGD adhesion motif, or/and a ligand selected from the group consisting of natural laminins, for example laminin-111, recombinant laminin isoforms, and biofunctional fragments thereof. Examples of suitable recombinant laminin isoforms are laminin-111, laminin-211, laminin-332, laminin-411, laminin-511, or laminin-521. An array of hydrogels varying in the above preselected features was established, by the method described above.

The mechanical properties of the hydrogels were also varied (hard (2000-3000 Pa), medium (1000-2000 Pa) or soft (50-1000 Pa) gels).

A variety of different known, commonly employed and/or commercially available culture media was used.

Tests were performed under hypoxic (low oxygen 5% O₂) or normoxic (18% O₂) conditions.

It was found that the presence of FBS (serum) or Wnt agonist such as R-spondin in the culture medium was favorable for cell growth. Also it was found that the hydrogel matrix should be preferably enzymatically-degradable.

In FIG. 5, the results of growth of different breast cancer cell types are shown. Brightfield images of human primary or metastatic (Mets) breast cancer cells from four patients of either HER2+ or Triple Negative Breast Cancer (TNBC) (from patient-derived xenograft models) are reproduced (4× objective magnification).

It can be seen in the bottom row that the hydrogel according to example 5a (non self-degradable, enzymatically degradable soft (<500 Pa) PEG hydrogel comprising a RGD motif and a laminin-111 as bioactive ligand) after the same time provided growth conditions similar to comparative example 5 (Matrigel®) in the upper row for TNBC lung metastatic cells and for TNBC primary cells.

The hydrogel according to example 5b (non self-degradable, enzymatically degradable soft (<500 Pa) PEG hydrogel comprising a RGD motif, but no laminin bioactive ligand) provided growth conditions similar to comparative example 5 (Matrigel®) for TNBC brain metastatic cells.

The hydrogel according to example 5c (non self-degradable, enzymatically degradable medium (>1000 Pa) PEG hydrogel comprising no RGD motif and no laminin bioactive ligand) provided growth conditions similar to comparative example 5 (Matrigel®) for HER2+ skin metastatic cells.

In general, the TNBC subtype was more challenging to grow. Hypoxic conditions improved breast cancer organoid growth over normoxic conditions. In addition, the morphology of HER2+ and TNBC cells grown under the preselected extracellular matrix conditions matched that of previously published breast cancer organoids established in Matrigel® (Sachs et al., 2018, Cell 172, 1-14).

### Example 6: Testing of Prostate Cancer Cells

Commercially available primary healthy prostate cells as well as prostate cancer cells from PDX cells were encapsulated within a range of different extracellular matrix conditions.

A PEG was used as a hydrogel precursor molecule to provide a non self-degradable hydrogel. As a crosslinking agent, peptides containing at least two, preferably two cysteine moieties were used which varied in their amino acid sequence, in particular with respect to the presence or absence of a RGD adhesion motif and the presence or absence of a MMP-sensitive motif.

A further variation that was made to some hydrogels was the attachment of a bioactive ligand comprising a RGD adhesion motif or/and a ligand selected from the group consisting of natural laminins, for example laminin-111, recombinant laminin isoforms, and biofunctional fragments thereof. Examples of suitable recombinant laminin isoforms are laminin-111, laminin-211, laminin-332, laminin-411, laminin-511, or laminin-521. An array of hydrogels varying in the above preselected features was established, by the method described above.

The mechanical properties of the hydrogels were also varied (hard (2000-3000 Pa), medium (1000-2000 Pa) or soft (50-1000 Pa) gels).

A variety of different known, commonly employed and/or commercially available culture media was used. Preferably, said culture medium is characterized by the presence of Wnt agonists such as R-spondin. According to a preferred embodiment, a culture medium may be used that is adapted from the medium described in Drost et al. (Nature Protocol 11, 347-358, January 2016) or Beshiri et al. (Clinical Cancer Research 24, 4332-4345), May 2018). The preferred culture medium comprises AdDMEM/F12 medium supplemented with glutamine, BSA, transferrin, Noggin, fibroblast growth factor 2 or basic, and FGF 10 [FGF2 or FGF-basic, and FGF10], EGF, R-spondin-conditioned medium, penicillin/streptomycin, glutathione, optionally N-acetyl-L-cysteine, Nicotinamide, DHT (dihydrotestosterone), insulin, prostaglandin E2, A83-01, SB202190 (p38-inhibitor), Y-27632 (rock inhibitor), and HEPES.

The results are shown in FIGS. 6a and 6b. The hydrogels in examples 6a and 6b were soft, enzymatically degradable hydrogels. Example 6a is a hydrogel that does not comprise a RGD motif. Example 6b is a hydrogel that comprises a RGD motif.

It was found that normal (healthy) cells were growing only in extracellular matrix containing the bioactive peptide RGD (FIG. 6a, Example 6b). Also, the growth of healthy cells was less pronounced with medium gels containing RGD compared to soft gels with RGD. On the other hand, in both examples 6a (without RGD) and 6b (with RGD) prostate cancer cells grew (FIG. 6b).

In contrast thereto, in the comparative example using naturally-derived matrix Matrigel®, no differentiation of growth of healthy prostate cells and prostate cancer cells could be achieved with either culture medium (FIGS. 6a and 6b).

1. A method to be performed with one tissue type, optionally, in combination with other cells including stromal cells or immune cells, comprising the steps of:
a) providing a fully defined hydrogel matrix array with discrete volumes by crosslinking, onto a substrate or into discrete volumes of a substrate, different combinations of one or more different hydrogel precursor molecules, optionally in the presence of one or more biologically active molecules, optionally at least one crosslinking agent and cells of the tissue type to be tested, so as to create fully defined three-dimensional extracellular matrix conditions that differ from each other in their biological, biophysical and/or biochemical characteristics;
b) allowing said cells to grow and expand in said discrete volumes of said hydrogel matrix array in the presence of one or more different culture media;
c) performing an operation with the cells grown in said discrete volumes of said hydrogel matrix array; and
wherein a specific combination of hydrogel features has been pre-selected for the said one tissue type to be tested.

2. The method according to claim 1, wherein the preselection of at least one of said hydrogel precursor molecules, or of said hydrogel features, and said culture media is made on the basis of selecting suitable extracellular matrix conditions from a method using random extracellular matrix conditions.

3. The method according to claim 1, wherein the tissue type is selected from the group consisting of cancer cells and normal/healthy cells.

4. The method according to claim 1, wherein freshly isolated or frozen cells from a biopsy or tissue resection of a human or from patient-derived xenograft (PDX) tissue are used.

5. The method according to claim 1, wherein in step c) one or more drugs are added to said discrete volumes of said hydrogel matrix.

6. The method according to claim 1, wherein the tissue type is lung cancer cells, overexpressing c-Met, and the hydrogel matrix is preselected as being a non self-degradable PEG hydrogel, wherein the crosslinking agent and said optional bioactive agent do not comprise any RGD motif.

7. The method according to claim 6, wherein said culture medium comprises FBS (serum) or Wnt agonist including R-spondin.

8. The method according to claim 1, wherein the tissue type is pancreatic ductal adenocarcinoma (PDAC) cells, and the hydrogel matrix is preselected as being a non self-degradable PEG hydrogel having a stiffness in the range of 50 to 3000 Pa, wherein at least one of the crosslinking agent and/or said optional bioactive agent comprise a RGD motif.

9. The method according to claim 8, wherein said culture medium comprises Wnt agonists including R-spondin and Wnt 3a.

10. The method according to claim 1, wherein the tissue type is colorectal cancer (CRC) cells, and the hydrogel matrix is preselected as being a PEG hydrogel having at least an initial stiffness in the range of 50 to 2000 Pa, and optionally furthermore comprising one or more biologically active molecules comprising laminin wherein at least one of the crosslinking agent and/or said optional bioactive agent comprise a RGD motif.

11. The method according to claim 10, wherein said culture medium comprises Wnt agonists including R-spondin and Wnt 3a.

12. The method according to claim 1, wherein the tissue type is breast cancer cells, and the hydrogel matrix is preselected as being an enzymatic-degradable PEG hydrogel, wherein at least one of the crosslinking agent comprises an enzymatically degradable motif, and said hydrogel optionally furthermore comprises one or more biologically active molecules comprising laminin.

13. The method according to claim 12, wherein said culture medium comprises FBS (serum) or Wnt agonist including R-spondin.

14. The method according to claim 1, wherein the tissue type is cancer cells that grow ex vivo more slowly than their normal counterparts, and the hydrogel matrix is preselected as being a PEG hydrogel, having a stiffness in the range of 50 to 2000 Pa, wherein said crosslinking agent and said optional bioactive agent do not comprise any RGD motif.

15. Kit of parts for performing an operation on or with one or more tissue type, comprising:
a) components for preparing a fully defined hydrogel matrix array, so as to create fully defined three-dimensional extracellular matrix conditions that differ from each other in their biological, biophysical and/or biochemical characteristics,
b) said components comprising
one or more different hydrogel precursor molecules,
optionally at least one crosslinking agent,
optionally one or more biologically active molecules,
c) one or more different culture media,
wherein a specific combination of hydrogel features has been pre-selected for the tissue type to be tested.

16. Kit according to claim 15, for testing the influence of drugs on lung cancer cells, overexpressing c-Met, the hydrogel matrix is preselected as being a non self-degradable PEG hydrogel, wherein the crosslinking agent and said optional bioactive agent do not comprise any RGD motif, and said culture medium comprises FBS (serum) or Wnt agonist including R-spondin.

17. Kit according to claim 15, for testing the influence of drugs on pancreatic ductal adenocarcinoma (PDAC) cells, the hydrogel matrix is preselected as being a non self-degradable PEG hydrogel having a stiffness in the range of 50 to 3000 Pa, wherein at least one of the crosslinking agent and/or said optional bioactive agent comprise a RGD motif, and said culture medium comprises Wnt agonists including R-spondin and Wnt 3a.

18. Kit according to claim 15, for testing the influence of drugs on colorectal cancer (CRC) cells, and the hydrogel matrix is preselected as being a PEG hydrogel having at least an initial stiffness in the range of 50 to 2000 Pa, and optionally furthermore comprising one or more biologically active molecules comprising laminin, wherein at least one of the crosslinking agent and/or said optional bioactive agent comprise a RGD motif, and wherein said culture medium comprises Wnt agonists including R-spondin and Wnt 3a.

19. Kit according to claim 15, for testing the influence of drugs on breast cancer cells, and the hydrogel matrix is preselected as being an enzymatic-degradable PEG hydrogel, wherein at least one of the crosslinking agent preferably comprises an enzymatically degradable motif, preferably a MMP sensitive motif, and said hydrogel optionally furthermore comprises one or more biologically active molecules comprising laminin, and wherein said culture medium comprises FBS (serum) or Wnt agonist including R-spondin.

20. Kit according to claim 15, for testing the influence of drugs on cancer cells that grow ex vivo more slowly than their normal counterparts, and the hydrogel matrix is preselected as being a PEG hydrogel, having a stiffness in the range of 50 to 2000 Pa, wherein said crosslinking agent and said optional bioactive agent do not comprise any RGD motif.
